SEQUENCE LISTING (15 sequences)

SEQ ID NO: 1 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 557 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 548 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 557 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 557 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 557 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 551 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 551 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 10 - protein, 564 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 11 - protein, 330 aa, Homo sapiens [sequence omitted]

SEQ ID NO: 12 - protein, 236 aa, Homo sapiens
PPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 13 - protein, 5 aa, Artificial
  Synthetic
GSGGS

SEQ ID NO: 14 - protein, 5 aa, Artificial
  Synthetic
GGGGS

SEQ ID NO: 15 - protein, 4 aa, Artificial
  Synthetic
GGGS

(19) United States
(12) Patent Application Publication
(10) Pub. No.: US 2010/0093979 A1
(43) Pub. Date: Apr. 15, 2010

(54) Fc Polypeptides With Novel Fc Ligand Binding Sites

(76) Inventor: Gregory Alan Lazar, (US)

Correspondence Address: MORGAN, LEWIS & BOCKIUS, LLP (SF), ONE MARKET SPEAR STREET TOWER, SAN FRANCISCO, CA, 94105, US

(21) Appl. No.: 12/578,360

(22) Filed: Oct. 13, 2009

Related U.S. Application Data
(63) Continuation of application No. 11/022,289, filed on Dec. 21, 2004, now abandoned.
(60) Provisional application No. 60/531,752, filed on Dec. 22, 2003.

(51) Int. Cl. C07K 16/00; C07H 21/00

(52) U.S. Cl. 530/387.3; 530/387.1; 536/23.53

(57) ABSTRACT

The present invention relates to Fc polypeptides with novel Fc receptor binding sites, and their application, particularly for therapeutic purposes.

This application is a continuation application of U.S. application Ser. No. 11/022,289, filed Dec. 21, 2004, and claims benefit under 35 U.S.C. §119(e) to U.S. Ser. No. 60/531,752 filed Dec. 22, 2003.

### FIELD OF THE INVENTION

The present invention relates to Fc polypeptides with novel Fc ligand binding sites, and their application, particularly for therapeutic purposes.

### BACKGROUND OF THE INVENTION

Antibodies and Fc fusions are common classes of therapeutic proteins that bind a specific antigen, and are used therapeutically for the treatment of a variety of conditions including cancer, inflammation, and cardiovascular disease. There are currently over ten antibody and Fc fusion products on the market, with numerous more in development. Despite such widespread use, these protein drugs are not optimized for clinical use. A significant deficiency of antibodies and Fc fusions is their suboptimal anticancer potency. Patient tumor response data show that monoclonal antibodies provide small to moderate improvements in therapeutic success over normal single-agent cytotoxic chemotherapeutics. The potency of antibodies as anti-cancer agents is unsatisfactory, and there is a substantial need to enhance the capacity of antibodies to destroy targeted cancer cells. Another property of antibodies and Fc fusions in need of improvement is their pharmacokinetics (PK). Despite long serum half-lives relative to small molecule drugs, the high cost and more demanding administration requirements mean that extension in the serum half-life of an antibody or Fc fusion translates directly into more effective and less expensive treatment. The present invention describes novel approaches to optimizing antibodies and Fc fusions for improved clinical properties, including but not limited to improvements in their cytotoxic capacity and pharmacokinetics.

In most mammals, including humans and mice, antibodies are constructed from paired heavy and light polypeptide chains. Each chain is made up of individual immunoglobulin (Ig) domains, and thus the generic term immunoglobulin is used for such proteins. The light and heavy chains are each made up of two distinct regions, referred to as the variable and constant regions. The variable regions show significant sequence diversity between antibodies, and are responsible for binding the target antigen. The constant regions show less sequence diversity, and are responsible for binding a number of natural proteins to elicit important biochemical events. In humans there are five different classes or isotypes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region. FIG. 1 shows an IgG1 antibody, used here as an example to describe the general structural features of antibodies. IgG antibodies are tetrameric proteins composed of two heavy chains and two light chains. The IgG heavy chain is composed of four immunoglobulin domains linked from N- to C-terminus in the order VH-CH1-CH2-CH3, referring to the variable heavy domain, constant heavy domain 1, constant heavy domain 2, and constant heavy domain 3. The IgG CH1, CH2, and CH3 domains are also referred to as Cγ1, Cγ2, and Cγ3 respectively. The IgG light chain is composed of two immunoglobulin domains linked from N- to C-terminus in the order VL-CL, referring to the variable light domain and the constant light domain respectively. A key feature of the antibody is the conserved N-linked glycosylation that occurs at aspargine 297 (Asn297). This carbohydrate, or oligosaccharide as it is sometimes referred, plays a critical structural and functional role for the antibody, and is one of the principle reasons that antibodies must be produced using mammalian expression systems.

In addition to antibodies, an antibody-like protein that is finding an expanding role in research and therapy is the Fc fusion (Chamow et al., 1996, Trends Biotechnol 14:52-60; Ashkenazi et al., 1997, Curr Opin Immunol 9:195-200). An Fc fusion is a protein wherein one or more polypeptides is operably linked to Fc. An Fc fusion combines the Fc region of an antibody, and thus its favorable effector functions and pharmacokinetics, with the target-binding region of a receptor, ligand, or some other protein or protein domain. The role of the latter is typically to mediate target recognition, and thus it is functionally analogous to the antibody variable region. Because of the structural and functional overlap of Fc fusions with antibodies, the discussion on antibodies in the present invention extends directly to Fc fusion.

The variable region of an antibody contains the antigen binding determinants of the molecule, and thus determines the specificity of an antibody for its target antigen. The variable region is so named because it is the most distinct in sequence from other antibodies within the same class. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain affinity and specificity for a broad array of antigens. A number of high-resolution structures are available for a variety of variable region fragments from different organisms, some unbound and some in complex with antigen. The sequence and structural features of antibody variable regions are well characterized (Morea et al., 1997, Biophys Chem 68:9-16; Morea et al., 2000, Methods 20:267-279), and the conserved features of antibodies have enabled the development of a wealth of antibody engineering techniques (Maynard et al., 2000, Annu Rev Biomed Eng 2:339-376). Fragments comprising the variable region can exist in the absence of other regions of the antibody, including for example the antigen binding fragment (Fab) comprising VH-CH1 and VL-CL, the variable fragment (Fv) comprising VH and VL, the single chain variable fragment (scFv) comprising VH and VL linked together in the same chain, as well as a variety of other variable region fragments (Little et al., 2000, Immunol Today 21:364-370).

The Fc region of an antibody or Fc fusion interacts with a number of Fc receptors and ligands, imparting an array of important functional capabilities referred to as effector functions. For IgG the Fc region comprises Ig domains CH2 and CH3 (Cγ2 and Cγ3) and the N-terminal hinge leading into CH2. An important family of Fc receptors for the IgG class is the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γγ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP).

A number of structures have been solved of the extracellular domains of human FcγRs, including FcγRIIa (pdb accession code 1H9V) (Sondermann et al., 2001, J Mol Biol 309:737-749) (pdb accession code 1FCG) (Maxwell et al., 1999, Nat Struct Biol 6:437-442), FcγRIIb (pdb accession code 2FCB) (Sondermann et al., 1999, Embo J 18:1095-1103); and FcγRIIIb (pdb accession code 1E4J) (Sondermann et al., 2000, Nature 406:267-273.). All FcγRs bind the same region on Fc, at the N-terminal end of the Cγ2 domain and the preceding hinge, shown in FIG. 2. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and 1IIX) (Radaev et al., 2001, J Biol Chem 276:16469-16477).

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical, however FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome.

FcγR-mediated effector functions have been implicated in the anti-cancer activity of antibodies, and a promising means for enhancing the anti-tumor potency of antibodies is via enhancement of their ability to mediate cytotoxic effector functions. There are a number of possible mechanisms by which antibodies destroy tumor cells, including anti-proliferation via blockage of needed growth pathways, intracellular signaling leading to apoptosis, enhanced down regulation and/or turnover of receptors, CDC, ADCC, ADCP, and promotion of an adaptive immune response (Cragg et al., 1999, Curr Opin Immunol 11:541-547; Glennie et al., 2000, Immunol Today 21:403-410). Anti-tumor efficacy may be due to a combination of these mechanisms, and their relative importance in clinical therapy appears to be cancer dependent. The importance of FcγR-mediated effector functions for the anti-cancer activity of antibodies has been demonstrated in mice (Clynes et al., 1998, Proc Natl Acad Sci USA 95:652-656; Clynes et al., 2000, Nat Med 6:443-446), and the affinity of interaction between Fc and certain FcγRs correlates with targeted cytotoxicity in cell-based assays (Shields et al., 2001, J Biol Chem 276:6591-6604; Shields et al., 2002, J Biol Chem 277:26733-26740) U.S. Pat. No. 6,737,056; U.S. Ser. No. 10/672,280; PCT/US03/30249; U.S. Ser. No. 10/822,231; U.S. Ser. No. 60/568,440; U.S. Ser. No. 60/627,026; U.S. Ser. No. 60/626,991; and U.S. Ser. No. 60/627,774). Additionally, a correlation has been observed between clinical efficacy in humans and their allotype of high (V158) or low (F158) affinity polymorphic forms of FcγRIIIa (Cartron et al., 2002, Blood 99:754-758) (Weng & Levy, 2003, Journal of Clinical Oncology, 21:3940-3947). Together these data suggest that an antibody that is optimized for binding to certain FcγRs may better mediate effector functions and thereby destroy cancer cells more effectively in patients. The balance between activating and inhibiting receptors is an important consideration, and optimal effector function may result from an antibody that has enhanced affinity for activation receptors, for example FcγRI, FcγRIIa/c, and FcγRIIIa, yet reduced affinity for the inhibitory receptor FcγRIIb. Furthermore, because FcγRs can mediate antigen uptake and processing by antigen presenting cells, enhanced FcγR affinity may also improve the capacity of antibody therapeutics to elicit an adaptive immune response.

Mutagenesis studies have been carried out on Fc towards various goals, with substitutions typically made to alanine (referred to as alanine scanning) or guided by sequence homology substitutions. The majority of substitutions reduce or ablate binding with FcγRs. However some success has been achieved at obtaining Fc variants with selectively enhanced binding to FcγRs, and in some cases these Fc variants have been shown to provide enhanced potency and efficacy in cell-based effector function assays. See for example U.S. Pat. No. 5,624,821, PCT WO 00/42072, U.S. Pat. No. 6,737,056, U.S. Ser. No. 10/672,280; PCT/US03/30249; U.S. Ser. No. 10/822,231; U.S. Ser. No. 60/568,440; U.S. Ser. No. 60/627,026; U.S. Ser. No. 60/626,991; and U.S. Ser. No. 60/627,774, and references cited therein. Enhanced affinity of Fc for FcγR has also been achieved using engineered glycoforms generated by expression of antibodies in engineered or variant cell lines (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473).

Optimization of complement-mediated effector functions also holds promise for improving the cytotoxic capacity of antibodies. CDC has been implicated as a component of the antibody therapeutic mechanism (Di Gaetano et al., 2003, J Immunol 171:1581-1587). A site on Fc overlapping with but separate from the FcγR binding site serves as the interface for the complement protein C1q. In the same way that Fc/FcγR binding mediates ADCC, Fc/C1q binding mediates complement dependent cytotoxicity (CDC). Clq forms a complex with the serine proteases C1r and C1s to form the C1 complex. C1q is capable of binding six antibodies, although binding to two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4 (Jefferis et al., 2002, Immunol Lett 82:57-65). There is currently no structure available for the Fc/C1q complex; however, mutagenesis studies have mapped the binding site on human IgG for C1q to a region involving residues D270, K322, K326, P329, and P331, and E333 (Idusogie et al., 2000, J Immunol 164:4178-4184; Idusogie et al., 2001, J Immunol 166:2571-2575). Mutagenesis aimed at enhancing the affinity of the antibody Fc region for C1q and enhancing CDC has met limited success (U.S. Pat. No. 6,737,056, PCT US2004/000643, U.S. Ser. No. 10/370,749, and PCT/US2004/005112; Idusogie et al., 2001, J. Immunology 166:2571-2572).

A critical parameter for the clinical efficacy of a protein therapeutic is its pharmacokinetics (PK). The longer the serum half-life of an antibody or Fc fusion, whether the therapeutic is used to treat cancer, auto-immune disease, inflammation, infectious disease, etc., the more time the drug has to carry out its intended function, and thus the better its efficacy. For Fc polypeptides such as antibodies and Fc fusions, PK is determined in part by the pH-dependant binding affinity of the Fc region for the neonatal receptor FcRn. The binding site for FcRn on IgG residues between the CH2 and CH3 domains. Binding of the receptor recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877) (FIG. 3), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481) provide insight into the interaction of Fc with these proteins. Several studies have shown that it is possible to engineer mutations in the Fc region that specifically enhance the pH-dependant affinity of an antibody for FcRn, in some cases resulting in improved serum half-life (Hinton et al., 2004, J. Biol. Chem. 279(8): 6213-6216; Dall' Acqua et al., 2002, J. Immuno. 169:5171-5180; Ghetie et al., 1997, Nat. Biotechnol. 15(7):637-640; WO2003US0033037; WO2004US0011213).

Taken together, the data suggest that the clinical properties of antibodies and Fc fusions may be optimized by modifying the binding of the Fc region to Fc ligands. Such modifications may enable improved clinical properties, including improved cell-mediated effector functions, improved complement-mediated effector functions, and improved pharmacokinetics. Despite progress, however, complete success has yet to be achieved, due in part to the incomplete understanding of the structural and functional determinants for these effector functions, as well as the difficulty in engineering variants with the desired Fc ligand specificity. In an embodiment, the present invention takes a novel approach to optimizing antibodies and Fc fusions. Provided herein are Fc polypeptides that comprise novel binding sites for Fc ligands. A number of methods and modifications are described for generating Fc polypeptides with novel Fc ligand binding sites that provide an array of optimized clinical properties. A variety of applications of the Fc polypeptides of the present invention are contemplated.

### SUMMARY OF THE INVENTION

It is an object of the present invention to provide Fc polypeptides that comprise one or more novel binding sites for one or more Fc ligands relative to a parent Fc polypeptide. An Fc polypeptide of the present invention comprises at least one additional Fc ligand binding site relative to its parent Fc polypeptide.

It is an object of the present invention to provide Fc polypeptides that comprise two or more Fc regions linked contiguously. In one embodiment, said Fc polypeptide comprises two or more Fc regions wherein all of the Fc regions composing the Fc polypeptide are of the same antibody isotype. In a preferred embodiment, said Fc polypeptide comprises two or more IgG Fc regions linked contiguously. In another embodiment, said Fc polypeptide comprises two or more Fc regions wherein two or more of the Fc regions composing the Fc polypeptide are of different antibody isotypes. In a preferred embodiment, said Fc polypeptide comprises one or more IgG Fc regions and one or more IgA Fc regions linked contiguously.

It is an object of the present invention to provide variant Fc polypeptides that comprise one or more novel binding sites for one or more Fc ligands relative to a parent Fc polypeptide. A variant Fc polypeptide of the present invention comprises one or more amino acid modifications relative to a parent Fc polypeptide, wherein said amino acid modification(s) provide or contribute to the binding of the Fc polypeptide to one or more Fc ligands. In a preferred embodiment, the Fc polypeptide of the invention comprises one or more amino acid modifications in an Fc region that enable the Fc polypeptide to bind to an Fc ligand that is not bound by the parent Fc polypeptide. In an alternately preferred embodiment, the variant Fc polypeptide binds to FcαRI and one or more FcγRs. In a most preferred embodiment, the Fc polypeptide is a variant of an IgG Fc polypeptide that comprises one or more amino acid modifications that enable the Fc polypeptide to bind FcαRI.

It is a further object of the present invention to provide methods for designing, engineering, producing, and experimentally testing and screening the Fc polypeptides.

It is an object of the present invention to provide isolated nucleic acids encoding the Fc polypeptides described herein. In an embodiment, the present invention provides vectors comprising said nucleic acids, optionally, operably linked to control sequences. It is an object of the present invention to provide host cells containing the vectors, and methods for producing and optionally recovering the Fc polypeptides.

It is an object of the present invention to provide novel antibodies and Fc fusions that comprise the Fc polypeptides disclosed herein. Said novel antibodies and Fc fusions may find use in a therapeutic product.

It is an object of the present invention to provide compositions comprising antibodies and Fc fusions that comprise the Fc polypeptides described herein, and a physiologically or pharmaceutically acceptable carrier or diluent.

The present invention contemplates therapeutic and diagnostic uses for antibodies and Fc fusions that comprise the Fc polypeptides disclosed herein.

### BRIEF DESCRIPTION OF THE DRAWINGS

FIG. 1. Antibody structure and function. Shown is a model of a full length human IgG1 antibody, modeled using a humanized Fab structure from pdb accession code 1CE1 (James et al., 1999, J Mol Biol 289:293-301) and a human IgG1 Fc structure from pdb accession code 1DN2 (DeLano et al., 2000, Science 287:1279-1283). The flexible hinge that links the Fab and Fc regions is not shown. IgG1 is a homodimer of heterodimers, made up of two light chains and two heavy chains. The Ig domains that comprise the antibody are labeled, and include VL and CL for the light chain, and VH Cgamma1 (Cγ1) (CH1), Cgamma2 (Cγ2) (CH2), and Cgamma3 (Cγ3) (CH3) for the heavy chain. The Fc region is labeled. Binding sites for relevant proteins are labeled, including the antigen binding site in the variable region, and the binding sites for FcγRs, FcRn, C1q, and proteins A and G in the Fc region. Attached carbohydrate are represented as black lines.

FIG. 2. The human IgG Fc/FcγRIII complex structure 1IIS (Radaev et al., 2001, J Biol Chem 276:16469-16477). Fc is shown as a black ribbon, and FcγRIII is shown as a grey ribbon. Attached carbohydrate are represented as black lines.

FIG. 3. The rat IgG Fc/FcRn complex structure 1I1A (Martin et al., 2001, Mol Cell 7:867-877). Fc is shown as a black ribbon diagram, and FcRn is shown as a grey ribbon. Attached carbohydrate are represented as black lines.

FIG. 4. Illustration of a homo-contiguously linked Fc polypeptide. Specifically, the protein is an FcgFcg polypeptide. CH2 and CH3 designate the Ig domains in the first Fc region, and CH2′ and CH3′ designate the Ig domains in the second Fc region. Hinge1 and hinge2 indicate the regions of the corresponding sequences provided in Example 1 and in FIG. 5.

FIG. 5. (SEQ ID NOS: 1-8). FcgFcg constructs described in Example 1. The constructs all have two contiguously linked gamma Fc regions, but differ in the hinge between the first and second Fc regions, i.e. hinge2. The hinge 1 sequence corresponds to the WT IgG1 hinge, whereas the hinge2 sequences correspond to the WT IgG1 hinge region or variants thereof.

FIG. 6. ALPHASCREEN™ assay showing binding of FcgFcg1 and FcgFcg2 polypeptides to human FcγRIIIa. The FcgFcg polypeptides comprise the variable regions of alemtuzumab. In the presence of competitor Fc polypeptide (WT alemtuzumab, FcgFcg1, or FcgFcg2) a characteristic inhibition curve is observed as a decrease in luminescence signal. BSA was used as the negative control. These data were normalized to the maximum and minimum luminescence signal provided by the baselines at low and high concentrations of competitor antibody respectively. The curves represent the fits of the data to a one site competition model using nonlinear regression.

FIG. 7. The human IgA Fc/FcαRI complex structure 1OW0 (Herr et al., 2003, Nature 423: 614-620). Fc is shown as a black ribbon, and FcαRI is shown as a grey ribbon. Attached carbohydrate are represented as black lines.

FIG. 8. Illustration of a hetero-contiguously linked Fc polypeptide. Specifically, the protein is an FcgFca polypeptide. CH2 and CH3 designate the Ig domains in the first Fc region, here an IgG1 Fc, and CH2′ and CH3′ designate the Ig domains in the second Fc region, here an IgA1 Fc. Hinge1 and hinge2 indicate the regions of the corresponding sequences provided in Example 2 and in FIG. 9.

FIG. 9. (SEQ ID NOS: 1, 9, 10). FcgFca construct FcgFca1 described in Example 2. The construct has an IgG1 Fc region linked contiguously to an IgA Fc region. The hinge 1 sequence corresponds to the WT IgG1 hinge, whereas the hinge2 sequence corresponds to the WT IgGA1 hinge region.

FIG. 10. ALPHASCREEN™ assay showing binding of human IgA and IgG antibodies to their respective human Fc receptors. FIG. 10a shows a dose response for binding of biotinylated-IgA streptavidin donor beads to GST-FcαRI glutathione acceptor beads. FIG. 10b shows a dose response for binding of biotinylated-IgG1 streptavidin donor beads to GST-FcγRIIIa (V158) glutathione acceptor beads. The data were normalized to the maximum and minimum luminescence signal provided by the baselines at low and high concentrations of competitor antibody respectively. The curves represent the fits of the data to a one site competition model using nonlinear regression.

FIG. 11. ALPHASCREEN™ assay showing binding of FcgFca1 with alemtuzumab variable regions to human V158 FcγRIIIa. In the presence of competitor FcgFca1 polypeptide, a characteristic inhibition curve is observed as a decrease in luminescence signal. The data were normalized to the maximum and minimum luminescence signal provided by the baselines at low and high concentrations of competitor antibody respectively. The curves represent the fits of the data to a one site competition model using nonlinear regression.

FIG. 12. The human IgA Fc/FcαRI binding interface (pdb accession code 1OW0; Herr et al., 2003, Nature 423: 614-620). Fc is shown as a grey ribbon, FcαRI is shown as a black ribbon, and residues on IgA Fc that mediate the interaction, as determined by visual inspection of the structure, are shown as black sticks.

FIG. 13. Sequence alignment of the human IgG1, IgA1, and IgA2 Fc regions, (SEQ ID NOS: 11, 9, 12). aligned using the sequence alignment program BLAST. IgG1 positions are numbered according to the EU index as in Kabat. Bold residues indicate the residues in the IgA Fc sequence, and the corresponding residues in IgG1 Fc, that mediate binding of IgA to FcαRI. FIG. 13a shows the alignment of the hinge and CH2 Ig domain, and FIG. 13b shows the alignment of the CH3 Ig domain. The 18 residues at the end of the IgA sequences not present in IgG1 represent the IgA tail piece.

FIG. 14. Structural superposition of the Fc regions of human IgG1 (black ribbon) and IgA1 (grey ribbon).

FIG. 15. Structure of the human IgA Fc/FcαRI binding interface (1OW0) showing glycosylation. IgA Fc is shown as a grey ribbon, and FcαRI is shown as a black ribbon. Carbohydrates attached to IgA Fc are shown as grey sticks, and carbohydrates attached to FcαRI are shown as black sticks.

FIG. 16. ALPHASCREEN™ assay showing binding of Fc variant trastuzumab antibodies to human V158 FcγRIIIa. In the presence of competitor antibody (WT or Fc variant trastuzumab) an inhibition curve is observed as a decrease in luminescence signal. BSA was used as the negative control. The data were normalized to the maximum and minimum luminescence signal provided by the baselines at low and high concentrations of competitor antibody respectively. The curves represent the fits of the data to a one site competition model using nonlinear regression.

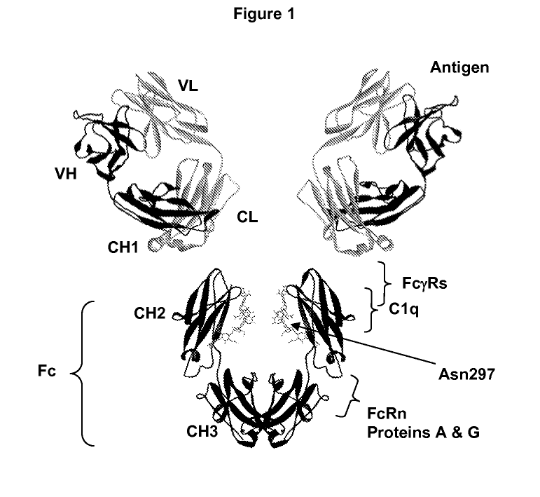

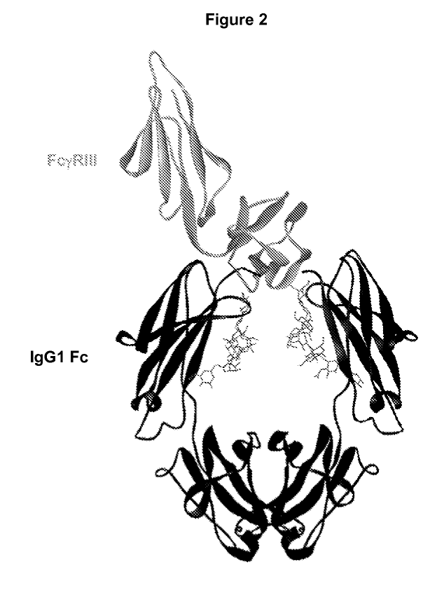

### DETAILED DESCRIPTION OF THE INVENTION

In order that the invention may be more completely understood, several definitions are set forth below. Such definitions are meant to encompass grammatical equivalents.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “amino acid modification” herein is meant an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution S426M refers to the substitution of methionine for serine at position 426.

By “antibody” herein is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)₂, Fv, scFv, or other antigen-binding subsequences of antibodies, either produced by the modification of whole antibodies or those synthesized de novo using recombinant DNA technologies.

Specifically included within the definition of “antibody” are full-length antibodies that contain an Fc region. By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, CH1, CH2, and CH3. In some mammals, for example in camels and llamas, full length IgG antibodies may consist of only two heavy chains, each heavy chain comprising a variable domain attached to the Fc region.

By “amino acid” and “amino acid identity” as used herein is meant one of the 20 naturally occurring amino acids or any non-natural analogs that may be present at a specific, defined polypeptide or protein position. Amino acids may be naturally occurring, or synthetic peptidomimetic structures, i.e. “analogs”, such as peptoids. The side chain may be in either the (R) or the (S) configuration. In the preferred embodiment, the amino acids are in the (S) or L-configuration.

By “effector function” as used herein is meant a biochemical event that results from the interaction of an antibody Fc region with an Fc receptor or Fc ligand. Effector functions include but are not limited to ADCC, ADCP, CDC, and FcRn-mediated serum half-life. By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γγ T cells, and may be from any organism including but not limited to humans, mice, rats, rabbits, and monkeys.

By “Fc region” as used herein is meant the polypeptide comprising the constant region of an antibody excluding the first constant region immunoglobulin domain. Fc region generally refers to the last two constant region immunoglobulin domains of IgA, IgD, and IgG, and the last three constant region immunoglobulin domains of IgE and IgM. Fc region may also include part or all of the flexible hinge N-terminal to these domains. For IgA and IgM, Fc region may or may not comprise the tailpiece, and may or may not be bound by the J chain. For IgG, Fc region comprises immunoglobulin domains Cgamma2 and Cgamma3 (Cγ2 and Cγ3) and the lower part of the hinge between Cgamma1 (Cγ1) and Cγ2. Although the boundaries of the Fc region may vary, the human IgG heavy chain Fc region is usually defined to comprise residues C226 or P230 to its carboxyl-terminus, wherein the numbering is according to the EU index as in Kabat. For IgA, Fc region comprises immunoglobulin domains Calpha2 and Calpha3 (Cα2 and Cα3) and the lower part of the hinge between Calpha1 (Cα1) and Cα2. Encompassed within the definition of Fc region are functionally equivalent analogs and variants of the Fc region. A functionally equivalent analog of Fc region may be a variant Fc region, comprising one or more amino acid modifications relative to the WT or naturally existing Fc region. Variant Fc regions will possess at least 50% homology with a naturally existing Fc region, with about 80% being preferred, and about 90% being more preferred, more preferably at least about 95% homology. Functionally equivalent analogs of Fc region may comprise one or more amino acid residues added to or deleted from the N- or C-termini of the protein, preferably no more than 30, most preferably no more than 10. Functionally equivalent analogs of Fc region include Fc regions operably linked to a fusion partner. Functionally equivalent analogs of Fc region must comprise the majority of all of the Ig domains that compose Fc region as defined above; for example IgG and IgA Fc regions as defined herein must comprise the majority of the sequence encoding CH2 and the majority of the sequence encoding CH3. Thus the CH2 domain on its own, or the CH3 domain on its own, are not considered Fc region in the present invention. Fc region may refer to this region in isolation, or this region in the context of an Fc polypeptide, as described below.

By “Fc polypeptide” as used herein is meant a polypeptide that comprises all or part of an Fc region. Fc polypeptides include antibodies, Fc fusions, isolated Fc regions, and functionally equivalent Fc analogs.

By “Fc fusion” as used herein is meant a protein wherein one or more polypeptides or small molecules is operably linked to an Fc region or derivative thereof. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200). An Fc fusion combines the Fc region of an immunoglobulin with a fusion partner, which in general can be any protein, polypeptide, peptide, or small molecule. The role of the non-Fc part of an Fc fusion, i.e., the fusion partner, is often but not always to mediate target binding, and thus it is functionally analogous to the variable regions of an antibody. A variety of linkers, defined and described below, may be used to covalently link an Fc region to a fusion partner to generate an Fc fusion.

By “Fc alpha receptor I” or “FcαRI” as used herein is meant any protein that binds the IgA antibody Fc region and is substantially encoded by an FcαRI gene (Otten & van Egmond, 2004, Immunology Letters 92:23-31). In humans this receptor includes but is not limited to FcαRI (CD89), FcαRI isoforms and allotypes, as well as any known or undiscovered human FcαRIs. An FcαRI may be from any organism, including but not limited to humans, mice, rats, rabbits, and monkeys.

By “Fc gamma receptor”, “FcγR” or “FcgR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRII) (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRII) (CD16), and FcγRII)-2 (CD16-2), as well as any undiscovered mouse FcγRs or FcγR isoforms or allotypes.

By “Fc ligand” or “effector ligand” as used herein is meant a polypeptide or other molecule from any organism that binds to an Fc region of an antibody to form an Fc/ligand complex (Jefferis et al., 2002, Immunol Lett 82:57-65). Fc ligands may bind to any antibody isotype, and include but are not limited to FcγRs (including but not limited to FcγRI, FcγRIIa, FcγRIIb, FcγRIIc, FcγRIIIa, FcγRIIIb, and allotypes thereof), FcαRs (including but not limited to FcαRI and allotypes thereof), FcεRs (including but not limited to FcεRI and allotypes thereof), Fc receptor homologs (FcRH) (including but not limited to FcRH1, FcRH2, FcRH3, FcRH4, FcRH5, and FcRH6) (Davis et al., 2002, Immunol. Reviews 190:123-136), FcRn, C1q, C3, FcαRI (CD89), Fcα/μ receptor, asialoglycoprotein-receptor (ASGP-R), transferrin receptor (TfR, CD71), secretory component (SC) receptor, M cell receptor, J chain, the polymeric Ig receptor involved in epithelial transport of IgA/IgM, mannan binding lectin, mannose receptor, staphylococcal protein A, streptococcal protein G, and viral Fc receptors. Fc ligands may include undiscovered molecules that bind Fc.

By “IgA” as used herein is meant a polypeptide belonging to the class of antibodies that are substantially encoded by a recognized immunoglobulin alpha gene. In humans this class or isotype comprises IgA1 and IgA2. IgA antibodies can exist as monomers, polymers (referred to as pIgA) of predominantly dimeric form, and secretory IgA. The constant chain of WT IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

By “IgG” as used herein is meant a polypeptide belonging to the class or isotype of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full length antibodies, antibody fragments, and individual immunoglobulin domains. Immunoglobulin heavy chains are grouped according to their “isotype” or “class”, as distinguished by the structure of their constant regions. The five main isotypes of immunoglobulin that are the antibody constant regions are IgM, IgD, IgG, IgE, and IgA. In humans, IgG immunoglobulins can be further subdivided into four subclasses (IgG1, IgG2, IgG3, and IgG4), whereas IgA immunoglobulins are found as two subclasses (IgA1 and IgA2).

By “immunoglobulin (Ig) domain” herein is meant a region of an immunoglobulin that exists as a distinct structural entity as ascertained by one skilled in the art of protein structure. Ig domains typically have a characteristic β-sandwich folding topology.

By “parent polypeptide” or “parent protein” as used herein is meant a polypeptide that is subsequently modified to generate a variant. Said parent polypeptide or protein may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant a Fc polypeptide that is modified to generate a variant, and by “parent antibody” as used herein is meant an antibody that is modified to generate a variant antibody.

By “protein” or “polypeptide” herein is meant at least two covalently attached amino acids, which includes proteins, polypeptides, oligopeptides and peptides. The protein may be made up of naturally occurring amino acids and peptide bonds, or synthetic peptidomimetic structures, i.e. analogs. By “single protein” or “single polypeptide” as used herein is meant a protein or polypeptide that contains only a contiguous sequence of amino acids, i.e. wherein all amino acid residues of the protein or polypeptide are linked via peptide bonds. Thus non-covalently linked polypeptides and polypeptides linked via covalent bonds other than peptide bonds, for example via disulfide bonds or post-translational modifications, are not herein considered single polypeptides.

By “position” as used herein is meant a location in the sequence of a protein. Positions may be numbered sequentially, or according to an established format, for example the EU index as in Kabat. For example, position 297 is a position in the human antibody IgG1. Corresponding positions are determined as outlined below, generally through sequence or structural alignment with other protein sequences.

By “residue” as used herein is meant a position in a protein and its associated amino acid identity. For example, Asparagine 297 (also referred to as Asn297, also referred to as N297) is a residue in the human antibody IgG1.

By “contiguously linked Fc polypeptide” (as well as grammatical equivalents) as used herein is meant an Fc polypeptide wherein two or more Fc regions are fused or linked. Contiguously linked Fc polypeptides may be homo- or hetero-contiguously linked, as described herein.

By “target antigen” as used herein is meant the molecule that is bound specifically by the variable region of a given antibody or Fc fusion. A target antigen may be a protein, carbohydrate, lipid, or other chemical compound. By “target cell” as used herein is meant a cell that expresses a target antigen.

By “variable region” as used herein is meant the region of an immunoglobulin that comprises one or more Ig domains substantially encoded by any of the VLκ, VLλ, and/or VH, chain genes that make up the light kappa, light lambda, and heavy chain immunoglobulin genetic loci respectively.

By “variant protein”, “protein variant”, “variant polypeptide”, or “polypeptide variant” as used herein is meant a polypeptide sequence that differs from that of a parent polypeptide sequence by virtue of at least one amino acid modification. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about twenty amino acid modifications, and preferably from about one to about ten amino acid modifications compared to the parent. The variant polypeptide sequence herein will preferably possess at least about 80% homology with a parent polypeptide sequence, and most preferably at least about 90% homology, more preferably at least about 95% homology. Accordingly, by “variant Fc” or “Fc variant” as used herein is meant an Fc sequence that differs from that of a parent Fc sequence by virtue of at least one amino acid modification. An Fc variant may only encompass an Fc region, or may exist in the context of an antibody, Fc fusion, or other polypeptide that is substantially encoded by Fc. Accordingly, by “variant Fc polypeptide” or “Fc polypeptide variant” as used herein is meant an Fc polypeptide, as defined above, that differs in sequence from that of a parent Fc polypeptide sequence by virtue of at least one amino acid modification. Variant Fc polypeptide may refer to the protein itself, compositions comprising the protein, or the amino acid sequence that encodes it.

For all immunoglobulin heavy chain constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda). The “EU index as in Kabat” refers to the residue numbering of the human IgG1 EU antibody.

### Fc Polypeptides of the Invention

In an embodiment, the present invention provides Fc polypeptides that comprise one or more novel binding sites for one or more Fc ligands relative to a parent Fc polypeptide. That is to say that an Fc polypeptide of the present invention, as defined herein, comprises at least one additional Fc ligand binding site relative to its parent Fc polypeptide. Novel Fc ligand binding sites may enable binding to any known or unknown Fc ligand or effector ligand, including but not limited to FcγRs, FcαRs, FcεRs, Fc receptor homologs, FcRH, FcRn, complement proteins, bacterial proteins A and G, and/or any Fc ligand as defined herein. Fc ligands may include undiscovered molecules that bind Fc.

The novel Fc ligand binding sites of the Fc polypeptides of the invention may provide an array of optimized properties. In a most preferred embodiment, the Fc polypeptides of the present invention provide optimized effector function properties relative to the parent. Properties that may be optimized include but are not limited to enhanced or reduced affinity for an Fc ligand. In one embodiment, engineered novel Fc ligand binding sites provide binding to an Fc ligand that is not bound by the parent Fc polypeptide. In an alternate embodiment, engineered novel Fc ligand binding sites provide binding to an Fc ligand that is already bound by the parent Fc polypeptide, i.e. the engineering of one or more novel Fc ligand binding sites serves to enhance binding of the Fc polypeptide to the Fc ligand by providing one or more additional binding sites to said Fc ligand. In a preferred embodiment, the Fc polypeptides of the present invention are optimized to possess enhanced affinity for a human activating FcγR, preferably FcγRI, FcγRIIa, FcγRIIc, FcγRIIIa, and FcγRIIIb, most preferably FcγRIIIa. In an alternately preferred embodiment, the Fc polypeptides are optimized to possess reduced affinity for the human inhibitory receptor FcγRIIb. In other embodiments, Fc polypeptides of the present invention provide enhanced affinity for one or more FcγRs, yet reduced affinity for one or more other FcγRs. For example, an Fc polypeptide of the present invention may have enhanced binding to FcγRIIIa, yet reduced binding to FcγRIIb. In a most preferred embodiment, the engineered novel Fc ligand binding sites provide the Fc polypeptide with binding to or enhanced binding to FcαRI. These preferred embodiments are anticipated to provide Fc polypeptides with enhanced cell-mediated effector functions, including but not limited to ADCC and ADCP. In alternately preferred embodiments, the engineered novel Fc ligand binding sites provide the Fc polypeptide with enhanced binding to one or more known or unknown complement proteins, for example C1q and C3. These preferred embodiments are anticipated to provide Fc polypeptides of the invention with enhanced complement-mediated effector functions relative to the parent Fc polypeptide, including but not limited to CDC. In alternately preferred embodiments, the engineered novel Fc ligand binding sites provide the Fc polypeptide of the invention with enhanced binding to FcRn, most preferably in a pH-dependant manner. This preferred embodiment is anticipated to provide Fc polypeptides of the invention with improved serum half-life and/or pharmacokinetics relative to the parent Fc polypeptide. In certain embodiments of the invention, IgA or IgM Fc regions may comprise their respective tail piece, and may be bound by the J chain. In these embodiments, the Fc polypeptides may provide novel and/or useful oligomerization and/or transport properties. All of the aforementioned embodiments are anticipated to provide Fc polypeptides of the invention with enhanced therapeutic properties in humans. Preferably, the Fc ligand specificity of the Fc polypeptide of the present invention will determine its therapeutic utility. The utility of a given Fc polypeptide for therapeutic purposes will depend also on the epitope or form of the target antigen and the disease or indication being treated.

Preferred embodiments comprise optimization of Fc binding to a human Fc ligands, however in alternate embodiments the Fc polypeptides of the present invention possess novel or enhanced binding to Fc ligands from nonhuman organisms, including but not limited to rodents and non-human primates. Fc polypeptides that are optimized for binding to a nonhuman Fc ligands may find use in experimentation. For example, mouse models are available for a variety of diseases that enable testing of properties such as efficacy, toxicity, and pharmacokinetics for a given drug candidate. As is known in the art, cancer cells can be grafted or injected into mice to mimic a human cancer, a process referred to as xenografting. Testing of Fc polypeptides that are optimized for binding to one or more mouse Fc ligands, may provide valuable information with regard to the efficacy of the protein, its mechanism of action, and the like.

In a preferred embodiment, additional Fc ligand binding sites are engineered via the generation of contiguously or contiguously linked Fc polypeptides. A contiguously or contiguously linked Fc polypeptide differs from its parent Fc polypeptide sequence in that the former comprises at least one additional Fc region relative to the latter. Contiguously or contiguously linked Fc polypeptides may be homo- or hetero-contiguously linked Fc polypeptides. Homo-contiguously linked Fc polypeptides comprise an Fc region of one isotype fused genetically to one or more Fc regions of the same isotype. Hetero-contiguously linked Fc polypeptides comprise an Fc region of one isotype fused genetically to one or more Fc regions of a different isotype. Any number of Fc regions from any of the recognized immunoglobulin constant region genes, including mu (μ), delta (δ), gamma (γ), sigma (σ), and alpha (α), which encode the IgM, IgD, IgG (including IgG1, IgG2, IgG3, and IgG4), IgE, and IgA (including IgA1 and IgA2) isotypes respectively, may be linked contiguously to generate a homo- or hetero-contiguously linked Fc polypeptide. Fc regions may be linked in any order, and any number of Fc regions may be linked contiguously. Functionally equivalent analogs of Fc regions may also find use in the present invention for generation of contiguously linked Fc polypeptides. The properties of any given contiguously linked Fc polypeptide will depend on the construct, and an array of valuable and unforeseen properties may be realized by combining Fc regions in various combinations using the concepts of engineering homo- and hetero-contiguously linked Fc polypeptides provided by the present invention.

In an alternately preferred embodiment, the engineering of additional Fc ligand binding sites is achieved via the engineering of variant Fc polypeptides. A variant Fc polypeptide comprises one or more amino acid modifications relative to a parent Fc polypeptide, wherein said amino acid modification(s) provide or contribute to the binding of the Fc polypeptide to one or more Fc ligands. Thus the Fc polypeptides of the present invention may be variant Fc polypeptides. An Fc polypeptide of the present invention differs in amino acid sequence from its parent Fc polypeptide by virtue of at least one amino acid modification. Thus variant Fc polypeptides of the present invention have at least one amino acid modification compared to the parent. Alternatively, the variant Fc polypeptides of the present invention may have more than one amino acid modification as compared to the parent, for example from about one to fifty amino acid modifications, preferably from about one to ten amino acid modifications, and most preferably from about one to about five amino acid modifications compared to the parent. Thus the sequences of the variant Fc polypeptides and those of the parent Fc polypeptides are substantially homologous. For example, the variant Fc polypeptide sequences herein will possess about 80% homology with the parent Fc polypeptide sequence, preferably at least about 90% homology, and most preferably at least about 95% homology.

The Fc polypeptides of the present invention may be an antibody, referred to herein as an antibody of the present invention. Antibodies of the present invention may comprise immunoglobulin sequences that are substantially encoded by immunoglobulin genes belonging to any of the antibody classes, including but not limited to IgG (including human subclasses IgG1, IgG2, IgG3, or IgG4), IgA (including human subclasses IgA1 and IgA2), IgD, IgE, IgG, and IgM classes of antibodies. Most preferably the antibodies of the present invention comprise sequences belonging to the human IgG and IgA classes of antibodies. The variable regions of any known or undiscovered antibody may find use in the present invention. Antibodies of the present invention may be nonhuman, chimeric, humanized, or fully human. As will be appreciated by one skilled in the art, these different types of antibodies reflect the degree of “humanness” or potential level of immunogenicity in a human. For a description of these concepts, see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). Said nonhuman variable region may be derived from any organism as described above, preferably mammals and most preferably rodents or primates. In one embodiment, the antibody of the present invention comprises monkey variable domains, for example as described in Newman et al., 1992, Biotechnology 10:1455-1460, U.S. Pat. No. 5,658,570, and U.S. Pat. No. 5,750,105. In a preferred embodiment, the variable region is derived from a nonhuman source, but its immunogenicity has been reduced using protein engineering. In a preferred embodiment, the antibodies of the present invention are humanized (Tsurushita & Vasquez, 2004, Humanization of Monoclonal Antibodies, Molecular Biology of B Cells, 533-545, Elsevier Science (USA)). By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. In a most preferred embodiment, the immunogenicity of the antibody has been reduced using a method described in U.S. Ser. No. 60/619,483, filed Oct. 14, 2004 and U.S. Ser. No. 11/004,590, entitled “Methods of Generating Variant Proteins with Increased Host String Content and Compositions Thereof”, filed on Dec. 3, 2004. In an alternate embodiment, the antibodies of the present invention may be fully human, that is the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

The Fc polypeptides of the present invention may be an Fc fusion, referred to herein as an Fc fusion of the present invention. Fc fusions of the present invention comprise an Fc polypeptide operably linked to one or more fusion partners. The role of the fusion partner typically, but not always, is to mediate binding of the Fc fusion to a target antigen. (Chamow et al., 1996, Trends Biotechnol 14:52-60; Ashkenazi et al., 1997, Curr Opin Immunol 9:195-200). Virtually any polypeptide or molecule that may serve as a fusion partner. Fc fusions of the invention may comprise immunoglobulin sequences that are substantially encoded by immunoglobulin genes belonging to any of the antibody classes, including but not limited to IgG (including human subclasses IgG1, IgG2, IgG3, or IgG4), IgA (including human subclasses IgA1 and IgA2), IgD, IgE, IgG, and IgM classes of antibodies. Most preferably the Fc fusions of the present invention comprise sequences belonging to the human IgG and IgA classes of antibodies.

Fc polypeptides of the present invention may be substantially encoded by genes from any organism, preferably mammals, including but not limited to humans, rodents including but not limited to mice and rats, lagomorpha including but not limited to rabbits and hares, camelidae including but not limited to camels, llamas, and dromedaries, and non-human primates, including but not limited to Prosimians, Platyrrhini (New World monkeys), Cercopithecoidea (Old World monkeys), and Hominoidea including the Gibbons and Lesser and Great Apes. In a most preferred embodiment, the Fc polypeptides of the present invention are substantially human. The Fc polypeptides of the present invention may be substantially encoded by immunoglobulin genes belonging to any of the antibody classes. In a most preferred embodiment, the Fc polypeptides of the present invention comprise sequences belonging to the IgG and IgA classes of antibodies. In an alternate embodiment, the Fc polypeptides of the present invention comprise sequences belonging to the IgD, IgE, IgG, or IgM classes of antibodies. The Fc polypeptides of the present invention may comprise more than one protein chain. That is, the present invention may find use in an Fc polypeptide that is a monomer or an oligomer, including a homo- or hetero-oligomer.

In the most preferred embodiment, the Fc polypeptides of the invention are based on human IgG1 and IgA1 sequences, and thus human IgG1 and IgA1 sequences are used as the “base” sequences against which other sequences are compared, including but not limited to sequences from other organisms, for example rodent and primate sequences, as well as sequences from other immunoglobulin classes such as IgE, IgGD, IgM, other IgG subclasses (for example IgG2, IgG3, and IgG4), other IgA subclasses (for example IgA2), and the like. It is contemplated that, although the Fc polypeptides of the present invention are engineered in the context of one parent Fc polypeptide, variants may be engineered in or “transferred” to the context of another, second parent Fc polypeptide. This is done by determining the “equivalent” or “corresponding” residues and substitutions between the first and second Fc polypeptides, typically based on sequence or structural homology between the sequences of the two Fc polypeptides. In order to establish homology, the amino acid sequence of a first Fc polypeptide outlined herein is directly compared to the sequence of a second Fc polypeptide. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first Fc polypeptide are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second Fc polypeptide that is at the level of tertiary structure for Fc polypeptides whose structures have been determined. In this case, equivalent residues are defined as those for which the atomic coordinates of two or more of the main chain atoms of a particular amino acid residue of the parent or precursor (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent Fc polypeptide in which the Fc polypeptides are made, what is meant to be conveyed is that the Fc polypeptides discovered by the present invention may be engineered into any second parent Fc polypeptide that has significant sequence or structural homology with said Fc polypeptide. Thus it is possible to use such methods to engineer amino acid modifications in an antibody or Fc fusion that comprise constant regions from other immunoglobulin classes, for example as described in U.S. Ser. Nos. 60/621,387, filed Oct. 21, 2004, and 60/629,068, filed Nov. 18, 2004, entitled “IgG Immunoglobulin Variants with Optimized Effector Function”. Thus for example, if a variant Fc polypeptide is generated wherein the parent polypeptide is a human IgG1 antibody, by using the methods described above or other methods for determining equivalent residues, said variant Fc polypeptide may be engineered in a human IgG2 parent antibody, a human IgA parent antibody, a mouse IgG2a or IgG2b parent antibody, and the like. Again, as described above, the context of the parent Fc polypeptide does not affect the ability to transfer the Fc polypeptides of the present invention to other parent Fc polypeptides. For example, a variant Fc polypeptide that is engineered in a human IgG1 antibody that targets one epitope may be transferred into a human IgG2 antibody that targets a different epitope, into an Fc fusion that comprises a human IgG1 Fc region that targets yet a different epitope, and so forth.

The Fc polypeptides of the present invention may find use in a wide range of products. In one embodiment the Fc polypeptide of the invention is a therapeutic, a diagnostic, or a research reagent, preferably a therapeutic. Alternatively, the Fc polypeptide of the present invention may be used for agricultural or industrial uses. An antibody of the present invention may find use in an antibody composition that is monoclonal or polyclonal. The Fc polypeptides of the present invention may be agonists, antagonists, neutralizing, inhibitory, or stimulatory. In a preferred embodiment, the Fc polypeptides of the present invention are used to kill target cells that bear the target antigen, for example cancer cells. In an alternate embodiment, the Fc polypeptides of the present invention are used to block, antagonize, or agonize the target antigen. In an alternately preferred embodiment, the Fc polypeptides of the present invention are used to block, antagonize, or agonize the target antigen and kill the target cells that bear the target antigen.

### Targets

Virtually any antigen may be targeted by the Fc polypeptides of the present invention, including but not limited to proteins, subunits, domains, motifs, and/or epitopes belonging to the following list of targets: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCl, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I(brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFα, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

One skilled in the art will appreciate that the aforementioned list of targets refers not only to specific proteins and biomolecules, but the biochemical pathway or pathways that comprise them. For example, reference to CTLA-4 as a target antigen implies that the ligands and receptors that make up the T cell co-stimulatory pathway, including CTLA-4, B7-1, B7-2, CD28, and any other undiscovered ligands or receptors that bind these proteins, are also targets. Thus target as used herein refers not only to a specific biomolecule, but the set of proteins that interact with said target and the members of the biochemical pathway to which said target belongs. One skilled in the art will further appreciate that any of the aforementioned target antigens, the ligands or receptors that bind them, or other members of their corresponding biochemical pathway, may be operably linked to the Fc polypeptides of the present invention in order to generate an Fc fusion. Thus for example, an Fc fusion that targets EGFR could be constructed by operably linking an Fc variant to EGF, TGF-β, or any other ligand, discovered or undiscovered, that binds EGFR. Accordingly, an Fc variant of the present invention could be operably linked to EGFR in order to generate an Fc fusion that binds EGF, TGF-β, or any other ligand, discovered or undiscovered, that binds EGFR. Thus virtually any polypeptide, whether a ligand, receptor, or some other protein or protein domain, including but not limited to the aforementioned targets and the proteins that compose their corresponding biochemical pathways, may be operably linked to the Fc polypeptides of the present invention to develop an Fc fusion.

A number of Fc polypeptides and Fc fusions that are approved for use, in clinical trials, or in development may benefit from the Fc polypeptides of the present invention. Thus in a preferred embodiment, the Fc polypeptides of the present invention may find use in a range of clinical products and candidates. The Fc polypeptides of the present invention may be incorporated into versions of clinical candidates and products that are humanized, affinity matured, engineered, or modified in some other way.

Choosing the right target antigen for antibody therapy is a complex process and encompasses many variables. For anti-cancer treatment it is desirable to have a target whose expression is restricted to the cancerous cells. Some targets that have proven especially amenable to antibody therapy are those with signaling functions. Other therapeutic antibodies exert their effects by blocking signaling of the receptor by inhibiting the binding between a receptor and it's cognate ligand. Another mechanism of action of therapeutic antibodies is to cause receptor down regulation. Although many therapeutically effective antibodies work in part by signaling through their target antigen, this is not always the case. For example, some target classes such as cell surface glycoforms do not generate any biological signal. However, altered glycoforms are often associated with disease states such as cancer. Another significant target type are those that internalize either as a normal function or in response to antibody binding. In the case of targets that are soluble rather than cell surface bound the recruitment of effector functions would not result in any cell death.

### Other Modifications

The Fc polypeptides of the present invention may be combined with other amino acid modifications in the Fc region that provide altered or optimized interaction with one or more Fc ligands, including but not limited to FcγRs, C1q, FcRn, FcR homologs, and/or as yet undiscovered Fc ligands. Additional modifications may provide altered or optimized affinity and/or specificity to the Fc ligands. Additional modifications may provide altered or optimized effector functions, including but not limited to ADCC, ADCP, CDC, and/or serum half-life. Such combination may provide additive, synergistic, or novel properties in antibodies or Fc fusions. In one embodiment, the Fc polypeptides of the present invention may be combined with known Fc variants. In a most preferred embodiment, the Fc polypeptides of the present invention comprise amino acid modifications that provide optimized effector function properties relative to the parent. Most preferred substitutions and optimized effector function properties are described in U.S. Ser. No. 10/672,280, PCT US03/30249, and U.S. Ser. No. 10/822,231, and U.S. Ser. No. 60/627,774, filed Nov. 12, 2004 and entitled “Optimized Fc Variants”. Alternate embodiments use other Fc modifications (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Lett 44:111-117; Lund et al., 1995, Faseb J 9:115-119; Jefferis et al., 1996, Immunol Lett 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490; Hinton et al., 2004, J Biol Chem 279:6213-6216) (U.S. Pat. No. 5,624,821; U.S. Pat. No. 5,885,573; U.S. Pat. No. 6,194,551; PCT WO 00/42072; PCT WO 99/58572; US 2004/0002587 A1), U.S. Pat. No. 6,737,056, PCT US2004/000643, U.S. Ser. No. 10/370,749, and PCT/US2004/005112). For example, as described in U.S. Pat. No. 6,737,056, PCT/US04/000643, U.S. Ser. No. 10/370,749, and PCT/US04/005112, the substitutions S298A, S298D, K326E, K326D, K326A, E333A, K334A, and P396L provide optimized FcγR binding and/or enhanced ADCC. Furthermore, as disclosed in Idusogie et al., 2001, J. Immunology 166:2571-2572, substitutions K326W, K326Y, and E333S provide enhanced binding to the complement protein C1q and enhanced CDC. Finally, as described in Hinton et al., 2004, J. Biol. Chem. 279(8): 6213-6216, substitutions T250Q, T250E, M428L, and M428F provide enhanced binding to FcRn and improved pharmacokinetics.

Because the binding sites for FcγRs, C1q, and FcRn reside in the Fc region, the differences between the IgGs in the Fc region are likely to contribute to differences in FcγR- and C1q-mediated effector functions. It is also possible that the modifications can be made in other non-Fc regions of an Fc polypeptide, including for example the Fab and hinge regions of an antibody, or the Fc fusion partner of an Fc fusion. For example, as disclosed in U.S. Ser. No. 60/556,353; U.S. Ser. No. 60/573,302; U.S. Ser. No. 60/585,328; U.S. Ser. No. 60/586,837; U.S. Ser. No. 60/589,906; U.S. Ser. No. 60/599,741; U.S. Ser. No. 60/607,398; U.S. Ser. No. 60/614,944; and U.S. Ser. No. 60/619,409, the Fab and hinge regions of an antibody may impact effector functions such as antibody dependent cell-mediated cytotoxicity (ADCC), antibody dependent cell-mediated phagocytosis (ADCP), and complement dependent cytotoxicity (CDC). Thus modifications outside the Fc region of an Fc polypeptide of the present invention are contemplated. For example, antibodies of the present invention may comprise one or more amino acid modifications in the VL, CL, VH, CH1, and/or hinge regions of an antibody.

The Fc polypeptides of the present invention may comprise modifications that modulate the in vivo pharmacokinetic properties of an Fc polypeptide. These include, but are not limited to, modifications that enhance affinity for the neonatal Fc receptor FcRn (U.S. Ser. No. 10/020,354; WO2001US0048432; EP2001000997063; U.S. Pat. No. 6,277,375; U.S. Ser. No. 09/933,497; WO1997US0003321; U.S. Pat. No. 6,737,056; WO2000US0000973; Shields et al. J. Biol. Chem., 276(9), 6591-6604 (2001); Zhou et al. J. Mol. Biol., 332, 901-913 (2003)). These further include modifications that modify FcRn affinity in a pH-specific manner. In some embodiments, where enhanced in vivo half-life is desired, modifications that specifically enhance FcRn affinity at lower pH (5.5-6) relative to higher pH (7-8) are preferred (Hinton et al. J. Biol. Chem. 279(8), 6213-6216 (2004); DaII' Acqua et al. J. Immuno. 169, 5171-5180 (2002); Ghetie et al. Nat. Biotechnol., 15(7), 637-640 (1997); PCT/US03/0033037; WO/US04/0011213). For example, as described in Hinton et al., 2004, “Engineered Human IgG Antibodies with Longer Serum Half-lives in Primates” J. Biol. Chem. 279(8): 6213-6216, substitutions T250Q, T250E, M428L, and M428F provide enhanced binding to FcRn and improved pharmacokinetics. Additionally preferred modifications are those that maintain the wild-type Fc's improved binding at lower pH relative to the higher pH. In alternative embodiments, where rapid in vivo clearance is desired, modifications that reduce affinity for FcRn are preferred. (U.S. Pat. No. 6,165,745; WO/US93/0003895; EP1993000910800; WO/US97/0021437; Medesan et al., J. Immunol., 158(5), 2211-2217 (1997); Ghetie and Ward, Annu. Rev. Immunol., 18, 739-766 (2000); Martin et al. Molecular Cell, 7, 867-877 (2001); Kim et al. Eur. J. Immunol. 29, 2819-2825 (1999)).

Fc polypeptides of the present invention may comprise one or more modifications that provide optimized properties that are not specifically related to effector function per se. Said modifications may be amino acid modifications, or may be modifications that are made enzymatically or chemically. Such modification(s) likely provide some improvement in the Fc polypeptide, for example an enhancement in its stability, solubility, function, or clinical use. The present invention contemplates a variety of improvements that made be made by coupling the Fc polypeptides of the present invention with additional modifications.

In a preferred embodiment, the Fc polypeptides of the present invention may comprise modifications to reduce immunogenicity in humans. In a most preferred embodiment, the immunogenicity of an Fc polypeptide of the present invention is reduced using a method described in U.S. Ser. No. 60/619,483, filed Oct. 14, 2004 and U.S. Ser. No. 11/004,590, entitled “Methods of Generating Variant Proteins with Increased Host String Content and Compositions Thereof”, filed on Dec. 3, 2004. In alternate embodiments, the antibodies of the present invention are humanized (Clark, 2000, Immunol Today 21:397-402). By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (see, e.g., Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; and U.S. Pat. No. 6,407,213). The humanized antibody optimally also will comprise at least a portion of an immunoglobulin constant region, typically that of a human immunoglobulin, and thus will typically comprise a human Fc region. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8. Humanization or other methods of reducing the immunogenicity of nonhuman antibody variable regions may include resurfacing methods, as described for example in Roguska et al., 1994, Proc. Natl. Acad. Sci. USA 91:969-973. In one embodiment, selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759. Other humanization methods may involve the grafting of only parts of the CDRs, including but not limited to methods described in U.S. Ser. No. 09/810,502; Tan et al., 2002, J. Immunol. 169:1119-1125; De Pascalis et al., 2002, J. Immunol. 169:3076-3084. Structure-based methods may be employed for humanization and affinity maturation, for example as described in U.S. Ser. No. 10/153,159 and related applications.

Modifications to reduce immunogenicity may include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an Fc polypeptide of the present invention. See for example WO 98/52976; WO 02/079232; WO 00/3317; U.S. Ser. No. 09/903,378; U.S. Ser. No. 10/039,170; U.S. Ser. No. 60/222,697; U.S. Ser. No. 10/339,788; PCT WO 01/21823; and PCT WO 02/00165; Mallios, 1999, Bioinformatics 15: 432-439; Mallios, 2001, Bioinformatics 17: 942-948; Sturniolo et al., 1999, Nature Biotech. 17: 555-561; WO 98/59244; WO 02/069232; WO 02/77187; Marshall et al., 1995, J. Immunol. 154: 5927-5933; and Hammer et al., 1994, J. Exp. Med. 180: 2353-2358. Sequence-based information can be used to determine a binding score for a given peptide-MHC interaction (see for example Mallios, 1999, Bioinformatics 15: 432-439; Mallios, 2001, Bioinformatics 17: p 942-948; Sturniolo et. al., 1999, Nature Biotech. 17: 555-561). It is possible to use structure-based methods in which a given peptide is computationally placed in the peptide-binding groove of a given MHC molecule and the interaction energy is determined (for example, see WO 98/59244 and WO 02/069232). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187). In a preferred embodiment, MHC-binding propensity scores are calculated for each 9-residue frame along the protein sequence using a matrix method (see Sturniolo et. al., supra; Marshall et. al., 1995, J. Immunol. 154: 5927-5933, and Hammer et. al., 1994, J. Exp. Med. 180: 2353-2358). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”. In an alternate embodiment, additional amino acid modifications may be engineered to reduce the propensity of the intact molecule to interact with B cell receptors and circulating antibodies.

Antibodies and Fc fusions of the present invention may comprise amino acid modifications in one or more regions outside the Fc region, for example the antibody Fab region or the Fc fusion partner, that provide optimal properties. In one embodiment, the variable region of an antibody of the present invention may be affinity matured, that is to say that amino acid modifications have been made in the VH and/or VL domains of the antibody to enhance binding of the antibody to its target antigen. Likewise, modifications may be made in the Fc fusion partner to enhance affinity of the Fc fusion for its target antigen. Such types of modifications may improve the association and/or the dissociation kinetics for binding to the target antigen. Other modifications include those that improve selectivity for target antigen vs. alternative targets. These include modifications that improve selectivity for antigen expressed on target vs. non-target cells. Other improvements to the target recognition properties may be provided by additional modifications. Such properties may include, but are not limited to, specific kinetic properties (i.e. association and dissociation kinetics), selectivity for the particular target versus alternative targets, and selectivity for a specific form of target versus alternative forms. Examples include full-length versus splice variants, cell-surface vs. soluble forms, selectivity for various polymorphic variants, or selectivity for specific conformational forms of the target.

Fc polypeptides of the invention may comprise one or more modifications that provide reduced or enhanced internalization of an Fc polypeptide. In one embodiment, Fc polypeptides of the present invention can be utilized or combined with additional modifications in order to reduce the cellular internalization of an Fc polypeptide that occurs via interaction with one or more Fc ligands. This property might be expected to enhance effector function, and potentially reduce immunogenicity of the Fc polypeptides of the invention. Alternatively, Fc polypeptides of the present Fc polypeptides of the present invention can be utilized directly or combined with additional modifications in order to enhance the cellular internalization of an Fc polypeptide that occurs via interaction with one or more Fc ligands. For example, in a preferred embodiment, an Fc polypeptide is used that provides enhanced binding to FcγRI, which is expressed on dendritic cells and active early in immune response. This strategy could be further enhanced by combination with additional modifications, either within the Fc polypeptide or in an attached fusion or conjugate partner, that promote recognition and presentation of Fc peptide fragments by MHC molecules. These strategies are expected to enhance target antigen processing and thereby improve antigenicity of the target antigen (Bonnerot and Amigorena, 1999, Immunol Rev. 172:279-84), promoting an adaptive immune response and greater target cell killing by the human immune system. These strategies may be particularly advantageous when the targeted antigen is shed from the cellular surface. An additional application of these concepts arises with idiotype vaccine immunotherapies, in which clone-specific antibodies produced by a patient's lymphoma cells are used to vaccinate the patient.

In a preferred embodiment, modifications are made to improve biophysical properties of the Fc polypeptides of the present invention, including but not limited to stability, solubility, and oligomeric state. Modifications can include, for example, substitutions that provide more favorable intramolecular interactions in the Fc polypeptide such as to provide greater stability, or substitution of exposed nonpolar amino acids with polar amino acids for higher solubility. A number of optimization goals and methods are described in U.S. Ser. No. 10/379,392 that may find use for engineering additional modifications to further optimize the Fc polypeptides of the present invention. The Fc polypeptides of the present invention can also be combined with additional modifications that reduce oligomeric state or size, such that tumor penetration is enhanced, or in vivo clearance rates are increased as desired.

Other modifications to the Fc polypeptides of the present invention include those that enable the specific formation or homodimeric or homomultimeric molecules. Such modifications include but are not limited to engineered disulfides, as well as chemical modifications or aggregation methods. Additional modifications to the variants of the present invention include those that enable the specific formation or heterodimeric, heteromultimeric, bifunctional, and/or multifunctional molecules. Such modifications include, but are not limited to, one or more amino acid substitutions in the CH3 domain, in which the substitutions reduce homodimer formation and increase heterodimer formation. For example, methods of engineering and compositions of such molecules are described in Atwell et al., 1997, J. Mol. Biol. 270(1):26-35, and Carter et al., 2001, J. Immunol. Methods 248:7-15. Additional modifications include modifications in the hinge and CH3 domains, in which the modifications reduce the propensity to form dimers.

In further embodiments, the Fc polypeptides of the present invention comprise modifications that remove proteolytic degradation sites. These may include, for example, protease sites that reduce production yields, as well as protease sites that degrade the administered protein in vivo. In a preferred embodiment, additional modifications are made to remove covalent degradation sites such as deamidation (i.e. deamidation of glutaminyl and asparaginyl residues to the corresponding glutamyl and aspartyl residues), oxidation, and proteolytic degradation sites. Deamidation sites that are particular useful to remove are those that have enhance propensity for deamidation, including, but not limited to asparaginyl and glutamyl residues followed by glycines (NG and QG motifs, respectively). In such cases, substitution of either residue can significantly reduce the tendency for deamidation. Common oxidation sites include methionine and cysteine residues. Other covalent modifications, that can either be introduced or removed, include hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group. Additional modifications also may include but are not limited to posttranslational modifications such as N-linked or O-linked glycosylation and phosphorylation.

Modifications may include those that improve expression and/or purification yields from hosts or host cells commonly used for production of biologics. These include, but are not limited to, various mammalian cell lines (e.g. CHO), yeast cell lines, bacterial cell lines, and plants. Additional modifications include modifications that remove or reduce the ability of heavy chains to form inter-chain disulfide linkages. Additional modifications include modifications that remove or reduce the ability of heavy chains to form intra-chain disulfide linkages.

The Fc polypeptides of the present invention may comprise modifications that include the use of unnatural amino acids incorporated using, for example, the technologies developed by Schultz and colleagues, including but not limited to methods described by Cropp & Shultz, 2004, Trends Genet. 20(12):625-30, Anderson et al., 2004, Proc. Natl. Acad. Sci. U.S.A. 101(2):7566-71, Zhang et al., 2003, 303(5656):371-3, and Chin et al., 2003, Science 301(5635):964-7. In some embodiments, these modifications enable manipulation of various functional, biophysical, immunological, or manufacturing properties discussed above. In additional embodiments, these modifications enable additional chemical modification for other purposes. For example, the Fc polypeptide may be linked to one of a variety of nonproteinaceous polymers, e.g., polyethylene glycol (PEG), polypropylene glycol, polyoxyalkylenes, or copolymers of polyethylene glycol and polypropylene glycol. Additional amino acid modifications may be made to enable specific or non-specific chemical or posttranslational modification of the Fc polypeptides. Such modifications, include, but are not limited to, PEGylation and glycosylation. Specific substitutions that can be utilized to enable PEGylation include, but are not limited to, introduction of novel cysteine residues or unnatural amino acids such that efficient and specific coupling chemistries can be used to attach a PEG or otherwise polymeric moiety. Introduction of specific glycosylation sites may be achieved by introducing novel N-X-T/S sequences into the Fc polypeptides of the present invention.

In one embodiment, the Fc polypeptides of the present invention comprise one or more engineered glycoforms. By “engineered glycoform” as used herein is meant a carbohydrate composition that is covalently attached to an Fc polypeptide, wherein said carbohydrate composition differs chemically from that of a parent Fc polypeptide. Engineered glycoforms may be useful for a variety of purposes, including but not limited to enhancing or reducing effector function. Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1); (Potelligent™ technology [Biowa, Inc., Princeton, N.J.]; GlycoMAb™ glycosylation engineering technology [Glycart Biotechnology AG, Zürich, Switzerland]). Many of these techniques are based on controlling the level of fucosylated and/or bisecting oligosaccharides that are covalently attached to the Fc region, for example by expressing an Fc polypeptide in various organisms or cell lines, engineered or otherwise (for example Lec-13 CHO cells or rat hybridoma YB2/0 cells), by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the Fc polypeptide has been expressed. Engineered glycoform typically refers to the different carbohydrate or oligosaccharide; thus an Fc polypeptide, for example an antibody or Fc fusion, may comprise an engineered glycoform. Alternatively, engineered glycoform may refer to the Fc polypeptide that comprises the different carbohydrate or oligosaccharide.

The Fc polypeptides of the present invention may be fused or conjugated to one or more other molecules or polypeptides. Conjugate and fusion partners may be any molecule, including small molecule chemical compounds and polypeptides. For example, a variety of antibody conjugates and methods are described in Trail et al., 1999, Curr. Opin. Immunol. 11:584-588. Possible conjugate partners include but are not limited to cytokines, cytotoxic agents, toxins, radioisotopes, chemotherapeutic agent, anti-angiogenic agents, a tyrosine kinase inhibitors, and other therapeutically active agents. In some embodiments, conjugate partners may be thought of more as payloads, that is to say that the goal of a conjugate is targeted delivery of the conjugate partner to a targeted cell, for example a cancer cell or immune cell, by the Fc polypeptide. Thus, for example, the conjugation of a toxin to an antibody or Fc fusion targets the delivery of said toxin to cells expressing the target antigen. As will be appreciated by one skilled in the art, in reality the concepts and definitions of fusion and conjugate are overlapping. The designation of an Fc polypeptide as a fusion or conjugate is not meant to constrain it to any particular embodiment of the present invention. Rather, these terms are used loosely to convey the broad concept that any Fc polypeptide of the present invention may be linked genetically, chemically, or otherwise, to one or more polypeptides or molecules to provide some desirable property.

In one embodiment, the Fc polypeptides of the present invention are fused or conjugated to a cytokine. By “cytokine” as used herein is meant a generic term for proteins released by one cell population that act on another cell as intercellular mediators. For example, as described in Penichet et al., 2001, J. Immunol. Methods 248:91-101, cytokines may be fused to antibody to provide an array of desirable properties. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; C5a; and other polypeptide factors including LIF and kit ligand (KL). As used herein, the term cytokine includes proteins from natural sources or from recombinant cell culture, and biologically active equivalents of the native sequence cytokines.

In an alternate embodiment, the Fc polypeptides of the present invention are fused, conjugated, or operably linked to a toxin, including but not limited to small molecule toxins and enzymatically active toxins of bacterial, fungal, plant or animal origin, including fragments and/or variants thereof. For example, a variety of immunotoxins and immunotoxin methods are described in Thrush et al., 1996, Ann. Rev. Immunol. 14:49-71. Small molecule toxins include but are not limited to calicheamicin, maytansine (U.S. Pat. No. 5,208,020), trichothene, and CC1065. In one embodiment of the invention, the antibody or Fc fusion is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody or Fc fusion (Chari et al., 1992, Cancer Research 52: 127-131) to generate a maytansinoid-antibody or maytansinoid-Fc fusion conjugate. Another conjugate of interest comprises an antibody or Fc fusion conjugated to one or more calicheamicin molecules. The calicheamicin family of antibiotics are capable of producing double-stranded DNA breaks at sub-picomolar concentrations. Structural analogs of calicheamicin that may be used include but are not limited to γ₁¹, α₂¹, α₃, N-acetyl-γ₁¹, PSAG, and Θ¹₁, (Hinman et al., 1993, Cancer Research 53:3336-3342; Lode et al., 1998, Cancer Research 58:2925-2928) (U.S. Pat. No. 5,714,586; U.S. Pat. No. 5,712,374; U.S. Pat. No. 5,264,586; U.S. Pat. No. 5,773,001). Dolastatin 10 analogs such as auristatin E (AE) and monomethylauristatin E (MMAE) may find use as conjugates for the Fc polypeptides of the present invention (Doronina et al., 2003, Nat Biotechnol 21(7):778-84; Francisco et al., 2003 Blood 102(4):1458-65). Useful enyzmatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, PCT WO 93/21232. The present invention further contemplates a conjugate between an Fc polypeptide of the present invention and a compound with nucleolytic activity, for example a ribonuclease or DNA endonuclease such as a deoxyribonuclease (Dnase).

In an alternate embodiment, an Fc polypeptide of the present invention may be fused, conjugated, or operably linked to a radioisotope to form a radioconjugate. A variety of radioactive isotopes are available for the production of radioconjugate antibodies and Fc fusions. Examples include, but are not limited to, At²¹¹, I¹³¹, I¹²⁵, Y⁹⁰, Re¹⁸⁶, Re¹⁸⁸, Sm¹⁵³, Bi²¹², P³², and radioactive isotopes of Lu.

In yet another embodiment, an Fc polypeptide of the present invention may be conjugated to a “receptor” (such streptavidin) for utilization in tumor pretargeting wherein the Fc polypeptide-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In an alternate embodiment, the Fc polypeptide is conjugated or operably linked to an enzyme in order to employ Antibody Dependent Enzyme Mediated Prodrug Therapy (ADEPT). ADEPT may be used by conjugating or operably linking the Fc polypeptide to a prodrug-activating enzyme that converts a prodrug (e.g. a peptidyl chemotherapeutic agent, see PCT WO 81/01145) to an active anti-cancer drug. See, for example, PCT WO 88/07378 and U.S. Pat. No. 4,975,278. The enzyme component of the immunoconjugate useful for ADEPT includes any enzyme capable of acting on a prodrug in such a way so as to covert it into its more active, cytotoxic form. Enzymes that are useful in the method of this invention include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuramimidase useful for converting glycosylated prodrugs into free drugs; beta-lactamase useful for converting drugs derivatized with .alpha.-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, for example, Massey, 1987, Nature 328: 457-458). Fc polypeptide-abzyme conjugates can be prepared for delivery of the abzyme to a tumor cell population. A variety of additional conjugates are contemplated for the Fc polypeptides of the present invention. A variety of chemotherapeutic agents, anti-angiogenic agents, tyrosine kinase inhibitors, and other therapeutic agents are described below, which may find use as Fc polypeptide conjugates.

Fusion and conjugate partners may be linked to any region of an Fc polypeptide of the present invention, including at the N- or C-termini, or at some residue in-between the termini. In a preferred embodiment, a fusion or conjugate partner is linked at the N- or C-terminus of the Fc polypeptide, most preferably the N-terminus. A variety of linkers may find use in the present invention to covalently link Fc polypeptides to a fusion or conjugate partner or generate an Fc fusion. By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 50 amino acid residues. Preferably, the linker is from about 1 to 30 amino acids in length, with linkers of 1 to 20 amino acids in length being most preferred. In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers (including, for example, (GS)n, (GSGGS)n (SEQ ID NO:13), (GGGGS)n (SEQ ID NO:14), and (GGGS)n (SEQ ID NO:15), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers may also be identified by screening databases of known three-dimensional structures for naturally occurring motifs that can bridge the gap between two polypeptide chains. In a preferred embodiment, the linker is not immunogenic when administered in a human patient. Thus linkers may be chosen such that they have low immunogenicity or are thought to have low immunogenicity. For example, a linker may be chosen that exists naturally in a human. In a most preferred embodiment, the linker has the sequence of the hinge region of an antibody, that is the sequence that links the antibody Fab and Fc regions; alternatively the linker has a sequence that comprises part of the hinge region, or a sequence that is substantially similar to the hinge region of an antibody. Another way of obtaining a suitable linker is by optimizing a simple linker, e.g., (Gly4Ser)n (SEQ ID NO:14), through random mutagenesis. Alternatively, once a suitable polypeptide linker is defined, additional linker polypeptides can be created to select amino acids that more optimally interact with the domains being linked. Other types of linkers that may be used in the present invention include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 1971, Science 238:1098. Chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody (see PCT WO 94/11026). The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the Fc polypeptides of the present invention to a fusion or conjugate partner to generate an Fc fusion, or to link the Fc polypeptides of the present invention to a conjugate.

### Experimental Production of Fc Polypeptides

In an embodiment, the present invention provides methods for producing and experimentally testing Fc polypeptides. The described methods are not meant to constrain the present invention to any particular application or theory of operation. Rather, the provided methods are meant to illustrate generally that one or more Fc polypeptides may be produced and experimentally tested to obtain variant Fc polypeptides. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76; Antibodies: A Laboratory Manual by Harlow & Lane, New York: Cold Spring Harbor Laboratory Press, 1988.

In one embodiment of the present invention, nucleic acids are created that encode the Fc polypeptides, and that may then be cloned into host cells, expressed and assayed, if desired. Thus, nucleic acids, and particularly DNA, may be made that encode each protein sequence. These practices are carried out using well-known procedures. For example, a variety of methods that may find use in the present invention are described in Molecular Cloning—A Laboratory Manual, 3^{rd} Ed. (Maniatis, Cold Spring Harbor Laboratory Press, New York, 2001), and Current Protocols in Molecular Biology (John Wiley & Sons). As will be appreciated by those skilled in the art, a variety of techniques that may be used to efficiently generate nucleic acids of the Fc polypeptides of the present invention. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode Fc polypeptides.

The Fc polypeptides of the present invention may be produced by culturing a host cell transformed with nucleic acid, preferably an expression vector, containing nucleic acid encoding the Fc polypeptides, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC® cell line catalog, available from the American Type Culture Collection.

In a preferred embodiment, the Fc polypeptides are expressed in mammalian expression systems, including systems in which the expression constructs are introduced into the mammalian cells using virus such as retrovirus or adenovirus. Any mammalian cells may be used, with human, mouse, rat, hamster, and primate cells being particularly preferred. Suitable cells also include known research cells, including but not limited to Jurkat T cells, NIH3T3, CHO, BHK, COS, HEK293, PER C.6, HeLa, Sp2/0, NS0 cells and variants thereof. In an alternately preferred embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, Fc polypeptides are produced in insect cells (e.g. Sf21/Sf9, Trichoplusia ni Bti-Tn5b1-4) or yeast cells (e.g. S. cerevisiae, Pichia, etc). In an alternate embodiment, Fc polypeptides are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the Fc polypeptides may be produced by chemical synthesis methods. Also transgenic expression systems both animal (e.g. cow, sheep or goat milk, embryonated hen's eggs, whole insect larvae, etc.) and plant (e.g. corn, tobacco, duckweed, etc.)

The nucleic acids that encode the Fc polypeptides of the present invention may be incorporated into an expression vector in order to express the protein. A variety of expression vectors may be utilized for protein expression. Expression vectors may comprise self-replicating extra-chromosomal vectors or vectors which integrate into a host genome. Expression vectors are constructed to be compatible with the host cell type. Thus expression vectors, which find use in the present invention, include but are not limited to those which enable protein expression in mammalian cells, bacteria, insect cells, yeast, and in in vitro systems. As is known in the art, a variety of expression vectors are available, commercially or otherwise, that may find use in the present invention for expressing Fc polypeptides.

Expression vectors typically comprise a protein operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the Fc polypeptide, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

Fc polypeptides may be operably linked to a fusion partner to enable targeting of the expressed protein, purification, screening, display, and the like. Fusion partners may be linked to the Fc polypeptide sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS (SEQ ID NO:14). A fusion partner may be a targeting or signal sequence that directs Fc polypeptide and any associated fusion partners to a desired cellular location or to the extracellular media. As is known in the art, certain signaling sequences may target a protein to be either secreted into the growth media, or into the periplasmic space, located between the inner and outer membrane of the cell. A fusion partner may also be a sequence that encodes a peptide or protein that enables purification and/or screening. Such fusion partners include but are not limited to polyhistidine tags (His-tags) (for example H₆ and H₁₀ or other tags for use with Immobilized Metal Affinity Chromatography (IMAC) systems (e.g. Ni⁺² affinity columns)), GST fusions, MBP fusions, Strep-tag, the BSP biotinylation target sequence of the bacterial enzyme BirA, and epitope tags which are targeted by antibodies (for example c-myc tags, flag-tags, and the like). As will be appreciated by those skilled in the art, such tags may be useful for purification, for screening, or both. For example, an Fc polypeptide may be purified using a His-tag by immobilizing it to a Ni⁺² affinity column, and then after purification the same His-tag may be used to immobilize the antibody to a Ni⁺² coated plate to perform an ELISA or other binding assay (as described below). A fusion partner may enable the use of a selection method to screen Fc polypeptides (see below). Fusion partners that enable a variety of selection methods are well-known in the art, and all of these find use in the present invention. For example, by fusing the members of an Fc polypeptide library to the gene III protein, phage display can be employed (Kay et al., Phage display of peptides and proteins: a laboratory manual, Academic Press, San Diego, Calif., 1996; Lowman et al., 1991, Biochemistry 30:10832-10838; Smith, 1985, Science 228:1315-1317). Fusion partners may enable Fc polypeptides to be labeled. Alternatively, a fusion partner may bind to a specific sequence on the expression vector, enabling the fusion partner and associated Fc polypeptide to be linked covalently or noncovalently with the nucleic acid that encodes them.

The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

In a preferred embodiment, Fc polypeptides are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of Fc polypeptides. For example, the bacterial proteins A and G bind to the Fc region. Likewise, the bacterial protein L binds to the Fab region of some antibodies, as of course does the antibody's target antigen. Purification can often be enabled by a particular fusion partner. For example, Fc polypeptides may be purified using glutathione resin if a GST fusion is employed, Ni⁺² affinity chromatography if a His-tag is employed, or immobilized anti-flag antibody if a flag-tag is used. For general guidance in suitable purification techniques, see Protein Purification: Principles and Practice, 3^{rd} Ed., Scopes, Springer-Verlag, NY, 1994. The degree of purification necessary will vary depending on the screen or use of the Fc polypeptides. In some instances no purification is necessary. For example in one embodiment, if the Fc polypeptides are secreted, screening may take place directly from the media. As is well known in the art, some methods of selection do not involve purification of proteins. Thus, for example, if a library of Fc polypeptides is made into a phage display library, protein purification may not be performed.

### Experimental Testing of Fc Polypeptides

### Assays

Fc polypeptides may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label. The use of fusion partners has been discussed above. By “labeled” herein is meant that the Fc polypeptides of the invention have one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and colorimetric dyes, or molecules such as biotin that enable other labeling methods. Labels may be incorporated into the compound at any position and may be incorporated in vitro or in vivo during protein expression.

In a preferred embodiment, the functional and/or biophysical properties of Fc polypeptides are screened in an in vitro assay. In vitro assays may allow a broad dynamic range for screening properties of interest. Properties of Fc polypeptides that may be screened include but are not limited to stability, solubility, and affinity for Fc ligands, for example FcγRs, FcαRs, FcRn, and the like. Multiple properties may be screened simultaneously or individually. Proteins may be purified or unpurified, depending on the requirements of the assay. In one embodiment, the screen is a qualitative or quantitative binding assay for binding of Fc polypeptides to a protein or nonprotein molecule that is known or thought to bind the Fc polypeptide. In a preferred embodiment, the screen is a binding assay for measuring binding to the target antigen. In an alternately preferred embodiment, the screen is an assay for binding of Fc polypeptides to an Fc ligand, including but are not limited to the family of FcγRs, FcαRs, the neonatal receptor FcRn, the complement protein C1q, and the bacterial proteins A and G. Said Fc ligands may be from any organism, with humans, mice, rats, rabbits, and monkeys preferred. Binding assays can be carried out using a variety of methods known in the art, including but not limited to FRET (Fluorescence Resonance Energy Transfer) and BRET (Bioluminescence Resonance Energy Transfer)-based assays, ALPHASCREEN™ (Amplified Luminescent Proximity Homogeneous Assay), Scintillation Proximity Assay, ELISA (Enzyme-Linked Immunosorbent Assay), SPR (Surface Plasmon Resonance, also known as BIACOR®), isothermal titration calorimetry, differential scanning calorimetry, gel electrophoresis, and chromatography including gel filtration. These and other methods may take advantage of some fusion partner or label of the Fc polypeptide. Assays may employ a variety of detection methods including but not limited to chromogenic, fluorescent, luminescent, or isotopic labels.

The biophysical properties of Fc polypeptides, for example stability and solubility, may be screened using a variety of methods known in the art. Protein stability may be determined by measuring the thermodynamic equilibrium between folded and unfolded states. For example, Fc polypeptides of the present invention may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an Fc polypeptide may be quantitatively or qualitatively determined using a wide range of methods that are known in the art. Methods which may find use in the present invention for characterizing the biophysical properties of Fc polypeptides include gel electrophoresis, isoelectric focusing, capillary electrophoresis, chromatography such as size exclusion chromatography, ion-exchange chromatography, and reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use. In one embodiment, stability and/or solubility may be measured by determining the amount of protein solution after some defined period of time. In this assay, the protein may or may not be exposed to some extreme condition, for example elevated temperature, low pH, or the presence of denaturant. Because function typically requires a stable, soluble, and/or well-folded/structured protein, the aforementioned functional and binding assays also provide ways to perform such a measurement. For example, a solution comprising an Fc polypeptide could be assayed for its ability to bind target antigen, then exposed to elevated temperature for one or more defined periods of time, then assayed for antigen binding again. Because unfolded and aggregated protein is not expected to be capable of binding antigen, the amount of activity remaining provides a measure of the Fc polypeptide's stability and solubility.

In a preferred embodiment, the library is screened using one or more cell-based or in vitro assays. For such assays, Fc polypeptides, purified or unpurified, are typically added exogenously such that cells are exposed to individual variants or groups of variants belonging to a library. These assays are typically, but not always, based on the biology of the ability of the Fc polypeptide to bind to the target antigen and mediate some biochemical event, for example effector functions like cellular lysis, phagocytosis, ligand/receptor binding inhibition, inhibition of growth and/or proliferation, apoptosis and the like. Such assays often involve monitoring the response of cells to Fc polypeptide, for example cell survival, cell death, cellular phagocytosis, cell lysis, change in cellular morphology, or transcriptional activation such as cellular expression of a natural gene or reporter gene. For example, such assays may measure the ability of Fc polypeptides to elicit ADCC, ADCP, or CDC. For some assays additional cells or components, that is in addition to the target cells, may need to be added, for example serum complement, or effector cells such as peripheral blood monocytes (PBMCs), NK cells, macrophages, and the like. Such additional cells may be from any organism, preferably humans, mice, rat, rabbit, and monkey. Crosslinked or monomeric antibodies and Fc fusions may cause apoptosis of certain cell lines expressing the antibody's target antigen, or they may mediate attack on target cells by immune cells which have been added to the assay. Methods for monitoring cell death or viability are known in the art, and include the use of dyes, fluorophores, immunochemical, cytochemical, and radioactive reagents. For example, caspase assays or annexin-fluorconjugates may enable apoptosis to be measured, and uptake or release of radioactive substrates (e.g. Chromium-51 release assays) or the metabolic reduction of fluorescent dyes such as alamar blue may enable cell growth, proliferation or activation to be monitored. In a preferred embodiment, the DELFIA® EuTDA-based cytotoxicity assay (Perkin Elmer, MA) is used. Alternatively, dead or damaged target cells may be monitored by measuring the release of one or more natural intracellular proteins, for example lactate dehydrogenase. Transcriptional activation may also serve as a method for assaying function in cell-based assays. In this case, response may be monitored by assaying for natural genes or proteins which may be upregulated or down-regulated, for example the release of certain interleukins may be measured, or alternatively readout may be via a luciferase or GFP-reporter construct. Cell-based assays may also involve the measure of morphological changes of cells as a response to the presence of an Fc polypeptide. Cell types for such assays may be prokaryotic or eukaryotic, and a variety of cell lines that are known in the art may be employed. Alternatively, cell-based screens are performed using cells that have been transformed or transfected with nucleic acids encoding the Fc polypeptides.

### Animal Models

The biological properties of the Fc polypeptides of the present invention may be characterized in cell, tissue, and whole organism experiments. As is know in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. Said animals may be referred to as disease models. With respect to the Fc polypeptides of the present invention, a particular challenge arises when using animal models to evaluate the potential for in-human efficacy of candidate polypeptides this is due, at least in part, to the fact that Fc polypeptides that have a specific effect on the affinity for a human Fc receptor may not have a similar affinity effect with the orthologous animal receptor. These problems can be further exacerbated by the inevitable ambiguities associated with correct assignment of true orthologues (Mechetina et al., Immunogenetics, 2002 54:463-468), and the fact that some orthologues simply do not exist in the animal (e.g. humans possess an FcγRIIa whereas mice do not). Therapeutics are often tested in mice, including but not limited to nude mice, SCID mice, xenograft mice, and transgenic mice (including knockins and knockouts). For example, an antibody or Fc fusion of the present invention that is intended as an anti-cancer therapeutic may be tested in a mouse cancer model, for example a xenograft mouse. In this method, a tumor or tumor cell line is grafted onto or injected into a mouse, and subsequently the mouse is treated with the therapeutic to determine the ability of the antibody or Fc fusion to reduce or inhibit cancer growth and metastasis. An alternative approach is the use of a SCID murine model in which immune-deficient mice are injected with human PBLs, conferring a semi-functional and human immune system—with an appropriate array of human FcRs—to the mice that have subsequently been injected with antibodies or Fc-polypeptides that target injected human tumor cells. In such a model, the Fc-polypeptides that target the desired antigen (such as her2/neu on SkOV3 ovarian cancer cells) interact with human PBLs within the mice to engage tumoricidal effector functions. Such experimentation may provide meaningful data for determination of the potential of said Fc polypeptide to be used as a therapeutic. Any organism, preferably mammals, may be used for testing. For example because of their genetic similarity to humans, monkeys can be suitable therapeutic models, and thus may be used to test the efficacy, toxicity, pharmacokinetics, or other property of the Fc polypeptides of the present invention. Tests of the Fc polypeptides of the present invention in humans are ultimately required for approval as drugs, and thus of course these experiments are contemplated. Thus the Fc polypeptides of the present invention may be tested in humans to determine their therapeutic efficacy, toxicity, pharmacokinetics, and/or other clinical properties.

Optimized Fc polypeptides can be tested in a variety of orthotopic tumor models. These clinically relevant animal models are important in the study of pathophysiology and therapy of aggressive cancers like pancreatic, prostate and breast cancer. Immune deprived mice including, but not limited to athymic nude or SCID mice are frequently used in scoring of local and systemic tumor spread from the site of intraorgan (e.g. pancreas, prostate or mammary gland) injection of human tumor cells or fragments of donor patients.

In preferred embodiments, Fc polypeptides of the present invention may be assessed for efficacy in clinically relevant animal models of various human diseases. In many cases, relevant models include various transgenic animals for specific tumor antigens. Relevant transgenic models such as those that express human Fc receptors (e.g., CD16 including the gamma chain, FcγRI, RIIa/b, and others) could be used to evaluate and test Fc polypeptide antibodies and Fc-fusions in their efficacy. The evaluation of Fc polypeptides by the introduction of human genes that directly or indirectly mediate effector function in mice or other rodents that may enable physiological studies of efficacy in tumor toxicity or other diseases such as autoimmune disorders and RA.

Because of the difficulties and ambiguities associated with using animal models to characterize the potential efficacy of candidate therapeutic antibodies in a human patient, some variant polypeptides of the present invention may find utility as proxies for assessing potential in-human efficacy. Such proxy molecules would preferably mimic—in the animal system—the Fc ligand biology of a corresponding candidate human Fc polypeptide. This mimicry is most likely to be manifested by relative association affinities between specific Fc polypeptides and animal vs. human Fc ligands.

In a preferred embodiment, the testing of Fc polypeptides may include study of efficacy in primates (e.g. cynomolgus monkey model) to facilitate the evaluation of depletion of specific target cells harboring target antigen. Additional primate models include but not limited to that of the rhesus monkey and Fc polypeptides in therapeutic studies of autoimmune, transplantation and cancer.

Toxicity studies are performed to determine the Fc polypeptide related-effects that cannot be evaluated in standard pharmacology profile or occur only after repeated administration of the agent. Most toxicity tests are performed in two species—a rodent and a non-rodent—to ensure that any unexpected adverse effects are not overlooked before new therapeutic entities are introduced into man. In general, these models may measure a variety of toxicities including genotoxicity, chronic toxicity, immunogenicity, reproductive/developmental toxicity and carcinogenicity. The general principles are that the products are sufficiently well characterized and for which impurities/contaminants have been removed, that the test material is comparable throughout development, and GLP compliance.

The pharmacokinetics (PK) of the Fc polypeptides of the invention can be studied in a variety of animal systems, with the most relevant being non-human primates such as the cynomolgus, rhesus monkeys. Single or repeated i.v./s.c. administrations over a dose range of 6000-fold (0.05-300 mg/kg) can be evaluated for the half-life (days to weeks) using plasma concentration and clearance as well as volume of distribution at a steady state and level of systemic absorbance can be measured. Examples of such parameters of measurement generally include maximum observed plasma concentration (Cmax), the time to reach Cmax (Tmax), the area under the plasma concentration-time curve from time 0 to infinity [AUC(0-inf] and apparent elimination half-life (T½). Additional measured parameters could include compartmental analysis of concentration-time data obtained following i.v. administration and bioavailability. Examples of pharmacological/toxicological studies using cynomolgus have been established for Rituxan and Zevalin in which monoclonal antibodies to CD20 are cross-reactive. Biodistribution, dosimetry (for radiolabeled antibodies or Fc fusions), and PK studies can also be done in rodent models. Such studies would evaluate tolerance at all doses administered, toxicity to local tissues, preferential localization to rodent xenograft animal models, depletion of target cells (e.g. CD20 positive cells).

The Fc polypeptides of the present invention may confer superior pharmacokinetics on Fc-containing therapeutics in animal systems or in humans. For example, increased binding to FcRn may increase the half-life and exposure of the Fc polypeptide drug. Alternatively, decreased binding to FcRn may decrease the half-life and exposure of the Fc polypeptide drug in cases where reduced exposure is favorable such as when such drug has side-effects.

It is known in the art that the array of Fc receptors is differentially expressed on various immune cell types, as well as in different tissues. Differential tissue distribution of Fc receptors may ultimately have an impact on the pharmacodynamic (PD) and pharmacokinetic (PK) properties of Fc polypeptides of the present invention. Because Fc polypeptides of the presentation may have varying affinities for the array of Fc receptors, further screening of the polypeptides for PD and/or PK properties may be extremely useful for defining the optimal balance of PD, PK, and therapeutic efficacy conferred by each candidate polypeptide. Pharmacodynamic studies may include, but are not limited to, targeting specific tumor cells or blocking signaling mechanisms, measuring depletion of target antigen expressing cells or signals, etc. The Fc polypeptides of the present invention may target particular effector cell populations and thereby direct Fc polypeptide drugs to recruit certain activities to improve potency or to increase penetration into a particularly favorable physiological compartment. Such pharmacodynamic effects may be demonstrated in animal models or in humans.

### Clinical Use of Fc Polypeptides

The Fc polypeptides of the present invention may be used for various therapeutic purposes. As will be appreciated by those in the art, the Fc polypeptides of the present invention may be used for any therapeutic purpose that antibodies, Fc fusions, and the like may be used for. In a preferred embodiment, the Fc polypeptides are administered to a patient to treat disorders including but not limited to autoimmune and inflammatory diseases, infectious diseases, and cancer.

A “patient” for the purposes of the present invention includes both humans and other animals, preferably mammals and most preferably humans. Thus the Fc polypeptides of the present invention have both human therapy and veterinary applications. The term “treatment” in the present invention is meant to include therapeutic treatment, as well as prophylactic, or suppressive measures for a disease or disorder. Thus, for example, successful administration of an Fc polypeptide prior to onset of the disease results in treatment of the disease. As another example, successful administration of an optimized Fc polypeptide after clinical manifestation of the disease to combat the symptoms of the disease comprises treatment of the disease. “Treatment” also encompasses administration of an optimized Fc polypeptide after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

### Diseases

In one embodiment, an Fc polypeptide of the present invention is administered to a patient having a disease involving inappropriate expression of a protein or other molecule. Within the scope of the present invention this is meant to include diseases and disorders characterized by aberrant proteins, due for example to alterations in the amount of a protein present, protein localization, posttranslational modification, conformational state, the presence of a mutant or pathogen protein, etc. Similarly, the disease or disorder may be characterized by alterations molecules including but not limited to polysaccharides and gangliosides. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the Fc polypeptides of the present invention.

By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

More particular examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g. nasopharyngeal cancer, salivary gland carcinoma, and esophageal cancer), lung (e.g. small-cell lung cancer, non-small cell lung cancer, adenocarcinoma of the lung and squamous carcinoma of the lung), digestive system (e.g. gastric or stomach cancer including gastrointestinal cancer, cancer of the bile duct or biliary tract, colon cancer, rectal cancer, colorectal cancer, and anal carcinoma), reproductive system (e.g. testicular, penile, or prostate cancer, uterine, vaginal, vulval, cervical, ovarian, and endometrial cancer), skin (e.g. melanoma, basal cell carcinoma, squamous cell cancer, actinic keratosis), liver (e.g. liver cancer, hepatic carcinoma, hepatocellular cancer, and hepatoma), bone (e.g. osteoclastoma, and osteolytic bone cancers) additional tissues and organs (e.g. pancreatic cancer, bladder cancer, kidney or renal cancer, thyroid cancer, breast cancer, cancer of the peritoneum, and Kaposi's sarcoma), and tumors of the vascular system (e.g. angiosarcoma and hemagiopericytoma).

By “autoimmune diseases” herein include allogenic islet graft rejection, alopecia greata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune dysfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erythematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis.

By “inflammatory disorders” herein include acute respiratory distress syndrome (ARDS), acute septic arthritis, allergic encephalomyelitis, allergic rhinitis, allergic vasculitis, allergy, asthma, atherosclerosis, chronic inflammation due to chronic bacterial or viral infections, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy.

By “infectious diseases” herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infections diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibria cholerae, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium mameffei, and the like. Infectious diseases may also be caused by protozoa and parasites such as chlamydia, kokzidioa, leishmania, malaria, rickettsia, trypanosoma, and the like.

Furthermore, Fc polypeptides of the present invention may be used to prevent or treat additional conditions including but not limited to heart conditions such as congestive heart failure (CHF), myocarditis and other conditions of the myocardium; skin conditions such as rosecea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological conditions.

### Formulation

Pharmaceutical compositions are contemplated wherein an Fc polypeptide of the present invention and one or more therapeutically active agents are formulated. Formulations of the Fc polypeptides of the present invention are prepared for storage by mixing said Fc polypeptide having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers antioxidants, preservatives, alkyl parabens, low molecular weight (less than about 10 residues) polypeptides; proteins, hydrophilic polymers, amino acids, monosaccharides, disaccharides, and other carbohydrates, chelating agents such as EDTA; sugars, sweeteners and other flavoring agents; fillers, binding agents, additives, coloring agents, salt-forming counter-ions, anionic, ionic and/or non-ionic surfactants, and PLURONIC® or polyethylene glycol (PEG). In a preferred embodiment, the pharmaceutical composition that comprises the Fc polypeptide of the present invention may be in a water-soluble form, such as being present as pharmaceutically acceptable salts, which is meant to include both acid and base addition salts. The formulations to be used for in vivo administration are preferably sterile. This is readily accomplished by filtration through sterile filtration membranes or other methods.

The Fc polypeptides disclosed herein may also be formulated as immunoliposomes. A liposome is a small vesicle comprising various types of lipids, phospholipids and/or surfactant that is useful for delivery of a therapeutic agent to a mammal. Liposomes containing the Fc polypeptide are prepared by methods known in the art, such as described in Epstein et al., 1985, Proc Natl Acad Sci USA, 82:3688; Hwang et al., 1980, Proc Natl Acad Sci USA, 77:4030; U.S. Pat. No. 4,485,045; U.S. Pat. No. 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The Fc polypeptide and other therapeutically active agents may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules.

### Administration

Administration of the pharmaceutical composition comprising an Fc polypeptide of the present invention, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, intracatherally, vaginally, parenterally, rectally, topically or intraocularly. In some instances, for example for the treatment of wounds, inflammation, etc., the Fc polypeptide may be directly applied as a solution or spray. As is known in the art, the pharmaceutical composition may be formulated accordingly depending upon the manner of introduction.

Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO 04091658). Antibodies or Fc fusions of the present invention may be more amenable to subcutaneous administration due to, for example, increased potency, improved serum half-life, or enhanced solubility.

As is known in the art, protein therapeutics are often delivered by IV infusion or bolus. The Fc polypeptides of the present invention may also be delivered using such methods. For example, administration may venous be by intravenous infusion with 0.9% sodium chloride as an infusion vehicle.

Pulmonary delivery may be accomplished using an inhaler or nebulizer and a formulation comprising an aerosolizing agent. For example, AERx® inhalable technology (Aradigm) or Inhance™ pulmonary delivery system (Nektar Therapeutics) may be used. Fc polypeptides of the present invention may be more amenable to intrapulmonary delivery. FcRn is present in the lung, and may promote transport from the lung to the bloodstream (e.g. Syntonix WO 04004798, Bitonti et. al. (2004) Proc. Nat. Acad. Sci. 101:9763-8). Accordingly, antibodies or Fc fusions that bind FcRn more effectively in the lung or that are released more efficiently in the bloodstream may have improved bioavailability following intrapulmonary administration. Fc polypeptides of the present invention may also be more amenable to intrapulmonary administration due to, for example, improved solubility or altered isoelectric point.

Furthermore, Fc polypeptides of the present invention may be more amenable to oral delivery due to, for example, improved stability at gastric pH and increased resistance to proteolysis. Furthermore, FcRn appears to be expressed in the intestinal epithelia of adults (Dickinson et. al. (1999) J. Clin. Invest. 104:903-11), so antibodies or Fc fusions of the present invention with improved FcRn interaction profiles may show enhanced bioavailability following oral administration. FcRn mediated transport of Fc polypeptides may also occur at other mucus membranes such as those in the gastrointestinal, respiratory, and genital tracts (Yoshida et. al. (2004) Immunity 20:769-83).

In addition, any of a number of delivery systems are known in the art and may be used to administer the Fc polypeptides of the present invention. Examples include, but are not limited to, encapsulation in liposomes, microparticles, microspheres (e.g. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers (e.g., Lupron Depot®, and poly-D-(−)-3-hydroxyburyric acid). It is also possible to administer a nucleic acid encoding the Fc polypeptide of the current invention, for example by retroviral infection, direct injection, or coating with lipids, cell surface receptors, or other transfection agents. In all cases, controlled release systems may be used to release the Fc polypeptide at or close to the desired location of action.

### Dosing

The dosing amounts and frequencies of administration are, in a preferred embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the therapeutically active Fc polypeptide in the formulation may vary from about 0.1 to about 100 weight %. In a preferred embodiment, the concentration of the Fc polypeptide is in the range of 0.003 to 1.0 molar. In order to treat a patient, a therapeutically effective dose of the Fc polypeptide of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from about 0.0001 to about 100 mg/kg of body weight or greater, for example about 0.1, 1, 10, or 50 mg/kg of body weight, with about 1 to about 10 mg/kg being preferred.

In some embodiments, only a single dose of the Fc polypeptide is used. In other embodiments, multiple doses of the Fc polypeptide are administered. In other embodiments the Fc polypeptides of the present invention are administered in metronomic dosing regimes, either by continuous infusion or frequent administration without extended rest periods. Such metronomic administration may involve dosing at constant intervals without rest periods. In certain embodiments the Fc polypeptide of the present invention and one or more other prophylactic or therapeutic agents are cyclically administered to the patient, as is known in the art. Cycling therapy may reduce the development of resistance to one or more agents, may minimize side effects, or may improve treatment efficacy.

### Combination Therapies

The Fc polypeptides of the present invention may be administered concomitantly with one or more other therapeutic regimens or agents. The additional therapeutic regimes or agents may be used to improve the efficacy or safety of the Fc polypeptide. Also, the additional therapeutic regimes or agents may be used to treat the same disease or a comorbidity rather than to alter the action of the Fc polypeptide. For example, an Fc polypeptide of the present invention may be administered to the patient along with chemotherapy, radiation therapy, or both chemotherapy and radiation therapy. The Fc polypeptide of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional Fc polypeptides, FcγRIIb or other Fc receptor inhibitors, or other therapeutic agents.

The terms “in combination with” and “co-administration” are not limited to the administration of said prophylactic or therapeutic agents at exactly the same time. Instead, it is meant that the Fc polypeptide of the present invention and the other agent or agents are administered in a sequence and within a time interval such that they may act together to provide a benefit that is increased versus treatment with only either the Fc polypeptide of the present invention or the other agent or agents. It is preferred that the Fc polypeptide and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

In one embodiment, the Fc polypeptides of the present invention are administered with one or more additional molecules comprising antibodies or Fc. The Fc polypeptides of the present invention may be co-administered with one or more other antibodies that have efficacy in treating the same disease or an additional comorbidity; for example two antibodies may be administered that recognize two antigens that are overexpressed in a given type of cancer, or two antigens that mediate pathogenesis of an autoimmune or infectious disease. The Fc polypeptides of the present invention may be co-administered with antibodies and/or Fc fusions that are used to treat any disease or indication, including but not limited to cancer, autoimmune disease, inflammatory disease, transplant rejection, GVHD, infectious diseases, and the like.

Alternatively, the Fc polypeptides of the present invention may be co-administered or with one or more other molecules that compete for binding to one or more Fc receptors. For example, co-administering inhibitors of the inhibitory receptor FcγRIIb may result in increased effector function. Similarly, co-administering inhibitors of the activating receptors such as FcγRIIIa may minimize unwanted effector function. Fc receptor inhibitors include, but are not limited to, Fc molecules that are engineered to act as competitive inhibitors for binding to FcγRIIb FcγRIIIa, or other Fc receptors, as well as other immunoglobulins and specifically the treatment called IVIg (intravenous immunoglobulin).

In one embodiment, the Fc polypeptides of the present invention are administered with a chemotherapeutic agent. By “chemotherapeutic agent” as used herein is meant a chemical compound useful in the treatment of cancer. Examples of chemotherapeutic agents include but are not limited to alkylating agents such as thiotepa and cyclosphosphamide (Cytoxan®), alkyl sulfonates such as busulfan, improsulfan and piposulfan; androgens such as calusterone, dromostanolone propionate, epitiostanol, mepitiostane, testolactone; anti-adrenals such as aminoglutethimide, mitotane, trilostane; anti-androgens such as flutamide, nilutamide, bicalutamide, leuprolide, and goserelin; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston); anti-metabolites such as methotrexate and 5-fluorouracil (5-FU); folic acid analogs such as denopterin, methotrexate, pteropterin, trimetrexate; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; folic acid replenisher such as frolinic acid; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; platinum analogs such as cisplatin and carboplatin; vinblastine; platinum; proteins such as arginine deiminase and asparaginase; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; taxanes, e.g. paclitaxel (Taxol®, Bristol-Myers Squibb) and docetaxel (Taxotere®, Rhone-Poulenc Rorer); topoisomerase inhibitor RFS 2000; thymidylate synthase inhibitor (such as Tomudex); additional chemotherapeutics including aceglatone; aldophosphamide glycoside; aminolevulinic acid; amsacrine; bestrabucil; bisantrene; edatraxate; defofamine; demecolcine; diaziquone; difluoromethylornithine (DMFO); elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; xeloda; ibandronate; CPT-11; retinoic acid; esperamicins; capecitabine. Pharmaceutically acceptable salts, acids or derivatives of any of the above may also be used.

A chemotherapeutic or other cytotoxic agent may be administered as a prodrug. By “prodrug” as used herein is meant a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.

A variety of other therapeutic agents may find use for administration with the Fc polypeptides of the present invention. In one embodiment, the Fc polypeptide is administered with an anti-angiogenic agent. By “anti-angiogenic agent” as used herein is meant a compound that blocks, or interferes to some degree, the development of blood vessels. The anti-angiogenic factor may, for instance, be a small molecule or a protein, for example an antibody, Fc fusion, or cytokine that binds to a growth factor or growth factor receptor involved in promoting angiogenesis. The preferred anti-angiogenic factor herein is an antibody that binds to Vascular Endothelial Growth Factor (VEGF). Other agents that inhibit signaling through VEGF may also be used, for example RNA-based therapeutics that reduce levels of VEGF or VEGF-R expression, VEGF-toxin fusions, Regeneron's VEGF-trap, and antibodies that bind VEGF-R. In an alternate embodiment, the Fc polypeptide is administered with a therapeutic agent that induces or enhances adaptive immune response, for example an antibody that targets CTLA-4. Additional anti-angiogenesis agents include, but are not limited to, angiostatin (plasminogen fragment), antithrombin III, angiozyme, ABT-627, Bay 12-9566, benefin, bevacizumab, bisphosphonates, BMS-275291, cartilage-derived inhibitor (CDI), CAI, CD59 complement fragment, CEP-7055, Col 3, combretastatin A-4, endostatin (collagen XVIII fragment), farnesyl transferase inhibitors, fibronectin fragment, gro-beta, halofuginone, heparinases, heparin hexasaccharide fragment, HMV833, human chorionic gonadotropin (hCG), IM-862, interferon alpha, interferon beta, interferon gamma, interferon inducible protein 10 (IP-10), interleukin-12, kringle 5 (plasminogen fragment), marimastat, metalloproteinase inhibitors (e.g. TIMPs), 2-methodyestradiol, MMI 270 (CGS 27023A), plasminogen activator inhibitor (PAI), platelet factor-4 (PF4), prinomastat, prolactin 16 kDa fragment, proliferin-related protein (PRP), PTK 787/ZK 222594, retinoids, solimastat, squalamine, SS3304, SU5416, SU6668, SU11248, tetrahydrocortisol-S, tetrathiomolybdate, thalidomide, thrombospondin-1 (TSP-1), TNP-470, transforming growth factor beta (TGF-β), vasculostatin, vasostatin (calreticulin fragment), ZS6126, and ZD6474.

In a preferred embodiment, the Fc polypeptide is administered with a tyrosine kinase inhibitor. By “tyrosine kinase inhibitor” as used herein is meant a molecule that inhibits to some extent tyrosine kinase activity of a tyrosine kinase. Examples of such inhibitors include but are not limited to quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lambert); antisense molecules (e.g. those that bind to ErbB-encoding nucleic acid); quinoxalines (U.S. Pat. No. 5,804,396); tryphostins (U.S. Pat. No. 5,804,396); ZD6474 (AstraZeneca); PTK-787 (Novartis/Schering A G); pan-ErbB inhibitors such as C1-1033 (Pfizer); Affinitac (ISIS 3521; Isis/Lilly); Imatinib mesylate (STI571, Gleevec®; Novartis); PKI 166 (Novartis); GW2016 (Glaxo SmithKline); C1-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; PCT WO 99/09016 (American Cyanamid); PCT WO 98/43960 (American Cyanamid); PCT WO 97/38983 (Warner-Lambert); PCT WO 99/06378 (Warner-Lambert); PCT WO 99/06396 (Warner-Lambert); PCT WO 96/30347 (Pfizer, Inc); PCT WO 96/33978 (AstraZeneca); PCT WO96/3397 (AstraZeneca); PCT WO 96/33980 (AstraZeneca), gefitinib (Iressa®, ZD1839, AstraZeneca), and OSI-774 (Tarceva®, OSI Pharmaceuticals/Genentech).

In another embodiment, the Fc polypeptide is administered with one or more immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but not limited to: non-steroidal anti-inflammatory drugs (NSAIDs) such as aspirin, ibuprofen, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (e.g. glucocorticoids, dexamethasone, cortisone, hydroxycortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFb, IFNa, IFNb, IFNg, IL-2, IL-4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromocryptine, cyclophosphamide, cyclosporine A, D-penicillamine, deoxyspergualin, FK506, sulfasasazine, glutaraldehyde, gold, hydroxychloroquine, leflunomide, malononitriloamides (e.g. leflunomide), methotrexate, minocycline, mizoribine, mycophenolate mofetil, and rapamycin.

In an alternate embodiment, Fc polypeptide of the present invention are administered with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of native sequence cytokines.

In a preferred embodiment, cytokines or other agents that stimulate cells of the immune system are co-administered with the Fc polypeptide of the present invention. Such a mode of treatment may enhance desired effector function. For example, agents that stimulate NK cells, including but not limited to IL-2 may be co-administered. In another embodiment, agents that stimulate macrophages, including but not limited to C5a, formyl peptides such as N-formyl-methionyl-leucyl-phenylalanine (Beigier-Bompadre et. al. (2003) Scand. J. Immunol. 57: 221-8), may be co-administered. Also, agents that stimulate neutrophils, including but not limited to G-CSF, GM-CSF, and the like may be administered. Furthermore, agents that promote migration of such immunostimulatory cytokines may be used. Also additional agents including but not limited to interferon gamma, IL-3 and IL-7 may promote one or more effector functions. In an alternate embodiment, cytokines or other agents that inhibit effector cell function are co-administered with the Fc polypeptide of the present invention. Such a mode of treatment may limit unwanted effector function.

In an additional embodiment, the Fc polypeptide is administered with one or more antibiotics, anti-fungal agents, and/or antiviral agents including protease inhibitors, reverse transcriptase inhibitors, and others, including type I interferons, viral fusion inhibitors, and neuramidase inhibitors.

The Fc polypeptides of the present invention may be combined with other therapeutic regimens. For example, in one embodiment, the patient to be treated with an antibody or Fc fusion of the present invention may also receive radiation therapy. Radiation therapy can be administered according to protocols commonly employed in the art and known to the skilled artisan. Such therapy includes but is not limited to cesium, iridium, iodine, or cobalt radiation. The radiation therapy may be whole body irradiation, or may be directed locally to a specific site or tissue in or on the body, such as the lung, bladder, or prostate. Typically, radiation therapy is administered in pulses over a period of time from about 1 to 2 weeks. The radiation therapy may, however, be administered over longer periods of time. For instance, radiation therapy may be administered to patients having head and neck cancer for about 6 to about 7 weeks. Optionally, the radiation therapy may be administered as a single dose or as multiple, sequential doses. The skilled medical practitioner can determine empirically the appropriate dose or doses of radiation therapy useful herein. In accordance with another embodiment of the invention, the Fc polypeptide of the present invention and one or more other anti-cancer therapies are employed to treat cancer cells ex vivo. It is contemplated that such ex vivo treatment may be useful in bone marrow transplantation and particularly, autologous bone marrow transplantation. For instance, treatment of cells or tissue(s) containing cancer cells with Fc polypeptide and one or more other anti-cancer therapies, such as described above, can be employed to deplete or substantially deplete the cancer cells prior to transplantation in a recipient patient.

Radiation therapy may also comprise treatment with an isotopically labeled molecule, such as an antibody. Examples of radioimmunotherapeutics include but are not limited to Zevalin® (Y⁹⁰ labeled anti-CD20), LymphoCide® (Y⁹⁰ labeled anti-CD22) and Bexxar® (I¹³¹ labeled anti-CD20).

It is of course contemplated that the Fc polypeptides of the invention may employ in combination with still other therapeutic techniques such as surgery or phototherapy.

In a preferred embodiment, patients are screened to predict the efficacy of the Fc polypeptides of the present invention. This information may be used, for example, to select patients to include or exclude from clinical trials or, post-approval, to provide guidance to physicians and patients regarding appropriate dosages and treatment options. Screening may involve the determination of the expression level or distribution of the target antigen. For example, the level of Her2/neu expression is currently used to select which patients will most favorably respond to trastuzumab therapy. Screening may also involve determination of genetic polymorphisms, for example polymorphisms related to Fc gamma and/or Fc alpha receptors. For example, patients who are homozygous or heterozygous for the F158 polymorphic form of FcγRIIIa may respond clinically more favorably to the Fc polypeptides of the present invention. Information obtained from patient screening may be used to select patients for inclusion in clinical trials, to determine appropriate dosages and treatment regimens, or for other clinical applications. Included in the present invention are diagnostic tests to identify patients who are likely to show a favorable clinical response to an Fc polypeptide of the present invention, or who are likely to exhibit a significantly better response when treated with an Fc polypeptide of the present invention versus one or more currently used biotherapeutics. Any of a number of methods for determining antigen expression levels, antigen distribution, and/or genetic polymorphisms in humans known in the art may be used.

Furthermore, the present invention comprises prognostic tests performed on clinical samples such as blood and tissue samples. Such tests may assay for effector function activity, including but not limited to ADCC, CDC, ADCP, phagocytosis, and opsonization, or for killing, regardless of mechanism, of cancerous or otherwise pathogenic cells. In a preferred embodiment, ADCC assays, such as those described previously, are used to predict, for a specific patient, the efficacy of a given Fc polypeptide of the present invention. Such information may be used to identify patients for inclusion or exclusion in clinical trials, or to inform decisions regarding appropriate dosages and treatment regemins. Such information may also be used to select a drug that contains a particular Fc polypeptide that shows superior activity in such assay.

### EXAMPLES

Examples are provided below to illustrate the present invention. These examples are not meant to constrain the present invention to any particular application or theory of operation.

For all immunoglobulin heavy chain positions discussed in the present invention, numbering is according to the EU index as in Kabat (Kabat et al., 1991, Sequences of Proteins of Immunological Interest, 5th Ed., United States Public Health Service, National Institutes of Health, Bethesda). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

### Example 1

### Homo-Contiguously Linked Fc Polypeptides

As described, there is a demand to improve the clinical properties of antibodies and Fc fusions. In an embodiment, the present invention provides Fc polypeptides with optimized properties wherein novel Fc receptor binding sites are engineered in a parent Fc polypeptide. In a preferred embodiment, the novel Fc polypeptides of the present invention comprise one or more additional Fc regions relative to a parent Fc polypeptide, thereby providing multiple binding sites for Fc receptors with a single protein molecule. Fc polypeptides with additional Fc receptor binding sites have been explored in the prior art. For example, multimeric Fc polypeptides have been engineered by linking Fab's and Fc's via thioether bonds originating at cysteine residues in the hinges. This chemical engineering approach has been used to generate molecules such as FabFc₂ (Kan et al., 2001, J. Immunol., 2001, 166: 1320-1326; Stevenson et al., 2002, Recent Results Cancer Res. 159: 104-12; U.S. Pat. No. 5,681,566). This chemical engineering strategy suffers, however, from problems of heterogeneity, production and purification, and potential immunogenicity. A more straightforward strategy is to contiguously link domains that comprise Fc ligand binding determinants. In one study, multiple Cγ2 domains have been fused between the Fab and Fc regions of an antibody (White et al., 2001, Protein Expression and Purification 21: 446-455; U.S. Ser. No. 10/096,521). This set of constructs may be suboptimal, however, with regard to structural and functional integrity, oligomerization, and potentially immunogenicity. Indeed the fusion of five Cγ2 domains resulted in only a two-fold enhancement in ADCC.

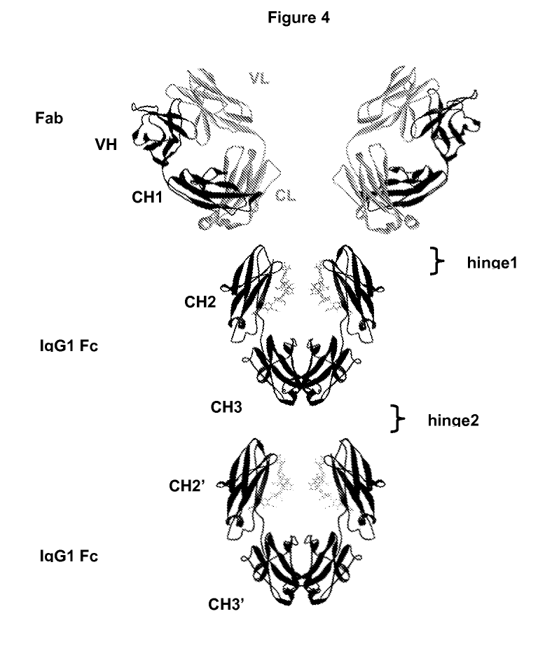

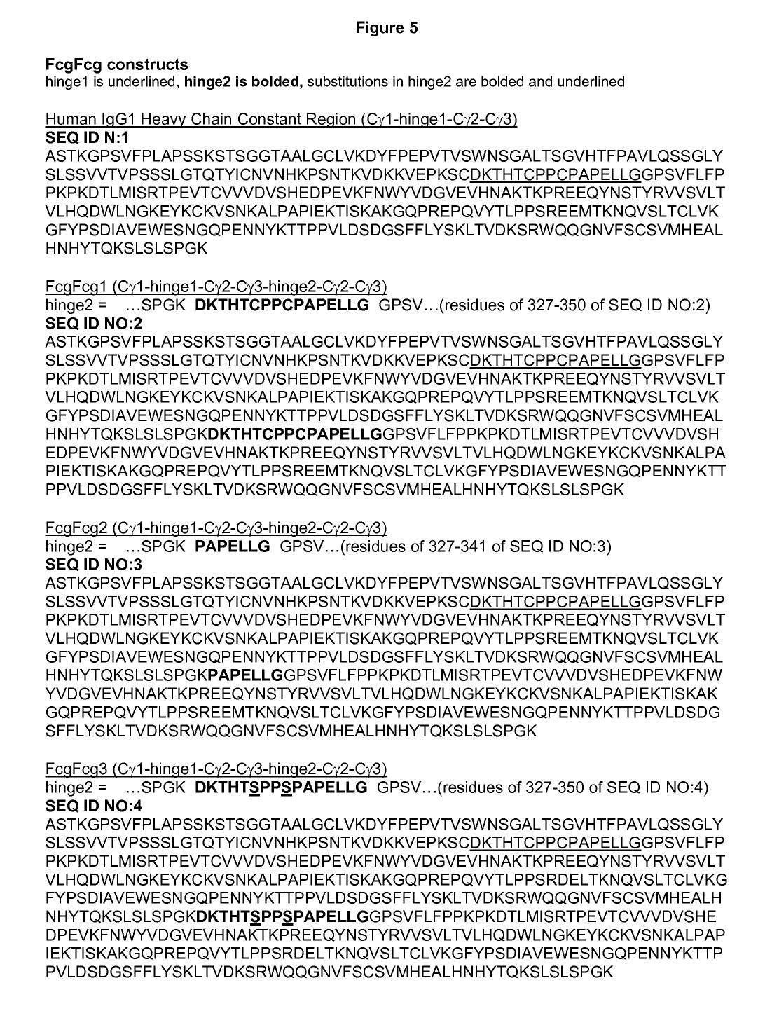

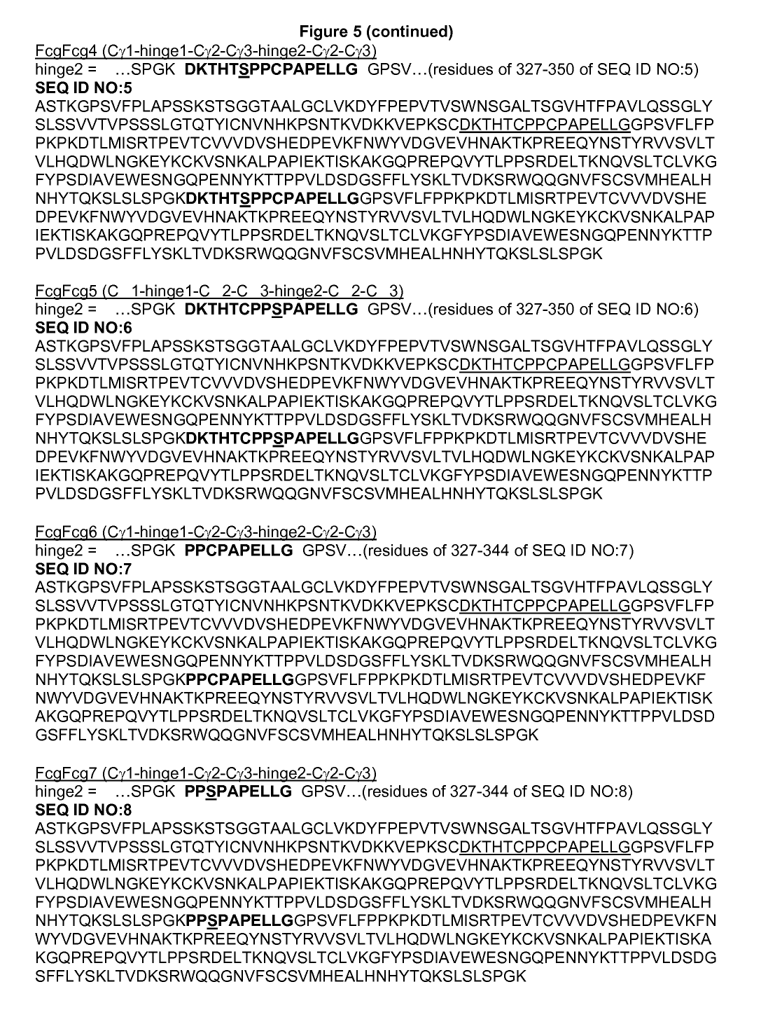

An embodiment of the present invention provides optimal Fc polypeptides with novel Fc ligand binding sites wherein the Fc region, not merely individual Ig domains, of one isotype is fused genetically to another Fc region of the same isotype. Such an engineered protein is herein referred to as a homo-contiguously linked Fc polypeptide. FIG. 4 illustrates this concept for a contiguously linked IgG Fc construct, referred to herein as FcgFcg. For the purposes of clarity, the set of Ig domains in the C-terminal Fc are referred to as CH2′ and CH3′. Thus in the embodiment provided by FIG. 4, CH2 and CH2′ are the IgG1 Cγ2 domain, and CH3 and CH3′ are the IgG1 Cγ3 domain. Hinge1, as designated in FIG. 4, links the Fab regions of the antibody with the first Fc region, whereas hinge2 links the first and second Fc regions. As will be appreciated by one skilled in the art, the hinge leading into the Fc region of an antibody plays an important structural and functional role. There are four cysteines that form two disulfides, providing an important structural constraint on the motion of the hinge, and thus on the antibody, Fc fusion, or other Fc polypeptide in general. Furthermore, residues in this hinge are involved in mediating binding to FcγRs. The optimal sequence of hinge2 may be determined by experimentation, and thus it may be prudent to explore a number of engineering constructs in order to obtain homo-contiguously linked Fc polypeptides with the most favorable structural and functional properties. FIG. 5 (SEQ ID NOS: 1-8) provides a number of genetic constructs aimed at engineering an effective FcgFcg. These constructs all have two contiguously linked gamma Fc regions, but differ in the hinge between the first and second Fc regions, i.e. hinge2. These hinges either correspond to the WT IgG1 hinge region or variants thereof, including modifications of the cysteines and/or truncations. The provision of these designed hinges are not meant to constrain the present invention, but rather to illustrate that the length and composition of the hinges (both hinge1 and hinge2) are important parameters for the contiguously linked Fc polypeptides of the present invention, and thus may be varied to achieve the optimal polypeptide. Linkers are preferably flexible and minimally immunogenic when administered in a human patient. A variety linker sequences, both natural and non-natural, are described above as potentially useful in the present invention for generating Fc fusions and conjugates. For example, rather than natural immunoglobulin hinge sequences, hinges may comprise glycine-serine polymers including, for example, (GS)n, (GSGGS)n (SEQ ID NO:13), (GGGGS)n (SEQ ID NO:14), and (GGGS)n (SEQ ID NO:15), where n is an integer of at least one), glycine-alanine polymers, alanine-serine polymers, or other flexible linkers. The particular linker sequences chosen for hinge1, hinge2, and subsequent hinges are not meant to constrain the invention.

FcgFcg1 and FcgFcg2 were constructed in the context of the variable regions of anti-CD52 antibody alemtuzumab (Campath®). Alemtuzumab is a humanized monoclonal antibody currently approved for treatment of B-cell chronic lymphocytic leukemia (Hale et al., 1990, Tissue Antigens 35:118-127). The genes for the variable regions of alemtuzumab were constructed using recursive PCR, and subcloned into a the mammalian expression vector pcDNA3.1Zeo (Invitrogen) comprising the full length light kappa (CLκ) and IgG1 heavy chain constant regions. FcgFcg1 and FcgFcg2 with the alemtuzumab variable region were constructed and subcloned into the pcDNA3.1Zeo vector using PCR. All genetic constructs were sequenced to confirm the fidelity of the sequence. Plasmids containing heavy chain genes were co-transfected with plasmid containing light chain genes into 293T cells. Media were harvested 5 days after transfection, and antibodies were purified from the supernatant using protein A affinity chromatography (Pierce).

In order to screen the Fc polypeptides for their capacity to bind FcγR, the extracellular region of human V158 FcγRIIIa was expressed and purified. The extracellular region of this receptor was obtained by PCR from a clone obtained from the Mammalian Gene Collection (MGC:22630), and fused with glutathione S-Transferase (GST) to enable screening. Tagged FcγRIIIa was transfected in 293T cells, and media containing secreted FcγRIIIa were harvested 3 days later and purified.

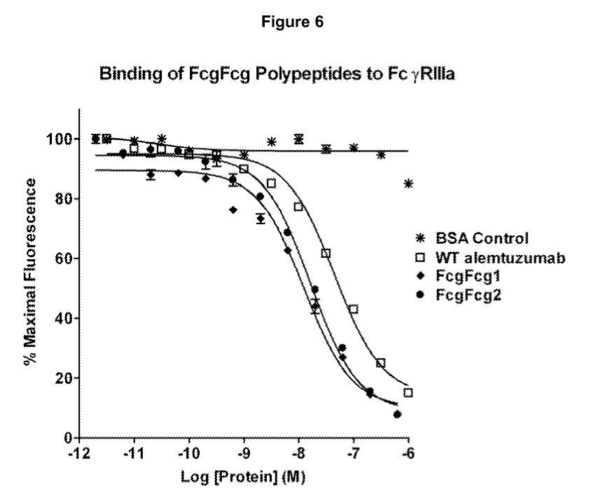

Binding affinity to human FcγRIIIa by WT alemtuzumab and the FcgFcg1 and FcgFcg2 polypeptides was measured using a quantitative and extremely sensitive method, ALPHASCREEN™ assay. The ALPHASCREEN™ assay is a bead-based non-radioactive luminescent proximity assay. Laser excitation of a donor bead excites oxygen, which if sufficiently close to the acceptor bead will generate a cascade of chemiluminescent events, ultimately leading to fluorescence emission at 520-620 nm. The ALPHASCREEN™ assay was applied as a competition assay for screening designed Fc polypeptides. WT IgG antibody was biotinylated by standard methods for attachment to streptavidin donor beads, and tagged FcγRIII (Val158 isoform) was bound to glutathione chelate acceptor beads. In the absence of competing Fc polypeptides, WT antibody and FcγR interact and produce a signal at 520-620 nm. Addition of untagged FcgFcg1 and FcgFcg2 competes with wild-type Fc/FcγR interaction, reducing fluorescence quantitatively to enable determination of relative binding affinities. FIG. 6 presents the ALPHASCREEN™ binding data for FcgFcg1 and FcgFcg2. As can be seen, FcgFcg1 and FcgFcg2 bind substantially more tightly to FcγRIIIa than WT alemtuzumab. These results indicate that engineering of the additional FcγR site in the contiguously linked polypeptides provides enhanced capacity to bind FcγRs; i.e. whereas WT antibody binds one FcγR per antibody, the contiguously linked Fc polypeptide co-engages two FcγRs simultaneously.

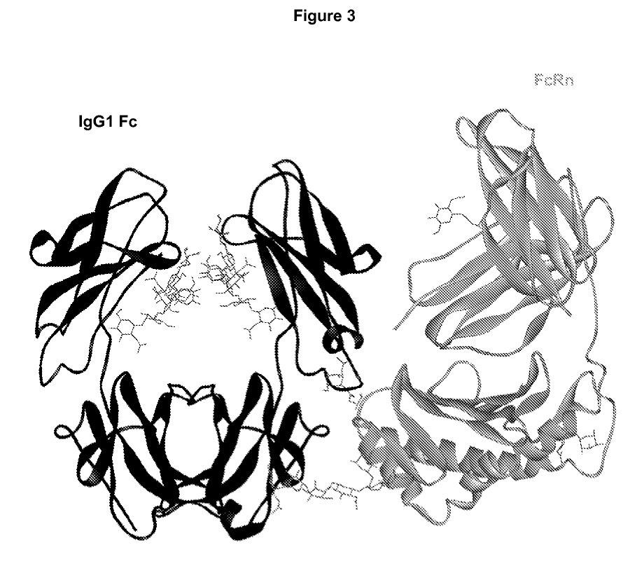

The enhanced FcγR binding provided by the homo-contiguously linked Fc polypeptides validates the engineering method, and indicates that it may be used to enhance the cytotoxic potency or other clinical properties of Fc polypeptides. By the same token, because FcgFcg1, FcgFcg2, and other contiguously linked Fc polypeptides provide additional binding sites for FcRn (see FIG. 3), the homo-contiguously linked Fc polypeptides of the present invention may provide enhanced binding to this receptor, improved serum half-life, and/or improved pharmacokinetics. Further experimentation of the FcgFcg and other homo-contiguously linked Fc polypeptides are contemplated. Binding studies to other Fc ligands may also be carried out, including but not limited to other FcγRs, complement protein C1q, FcRn, and protein A. Cell-based assays may be used to evaluate the capacity of the variants to mediate effector functions. Pre-clinical and clinical experiments may ultimately be used to evaluate the potential of the variant Fc polypeptides for therapeutic use.

### Example 2

### Hetero-Contiguously Linked Fc Polypeptides

Although IgG is the principal antibody isoform used for therapeutic applications, other isoforms have therapeutic potential. In particular, recent evidence indicates that IgA Fc ligands can initiate a number of potent effector functions, including endocytosis, phagocytosis, antibody-dependent cellular cytotoxicity (ADCC), antigen presentation, and release of inflammatory mediators, challenging the view of IgA as a non-inflammatory antibody (van Egmond et al., 2001, Trends in Immunology, 22: 205-210; Otten & van Egmond, 2004, Immunology Letters 92:23-31). IgA is the most prominent isotype of antibodies at mucosal surfaces, and the second most predominant isotype in human serum. IgA antibodies can exist as monomers, polymers (referred to as pIgA) of predominantly dimeric form, and secretory IgA. The constant chain of WT IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece. The polymeric immunoglobulin receptor (pIgR) expressed by mucosal and glandular epithelial cells binds the submucosally produced pIgA and transports the pIgA from the basolateral surface to the apical surface in contact with external secretions. At the apical surface the ectoplasmic domain, also known as the secretory component (SC), is cleaved from the transmembrane domain.

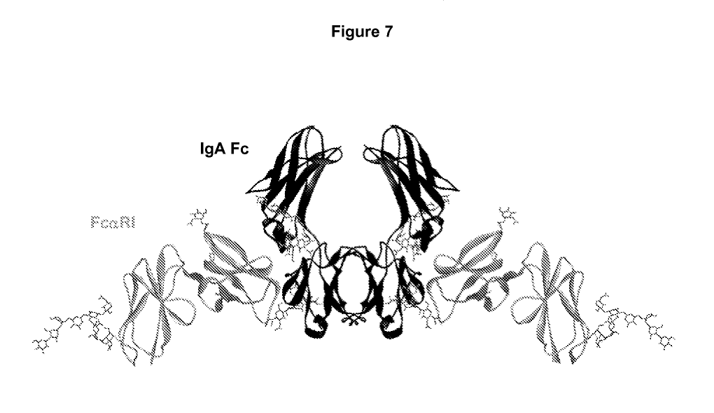

Several Fc ligands for IgA have been described, including the myeloid IgA Fc receptor, FcαRI (CD89), Fcα/μ, receptor, asialoglycoprotein-receptor (ASGP-R), transferrin receptor (TfR, CD71), secretory component (SC) receptor, M cell receptor, and polymeric Ig receptor, which can bind to the Fc tail, IgA carbohydrate side chains or to accessory molecules such as the J-chain and SC. The most well-characterized of these is FcαRI (Otten & van Egmond, 2004, Immunology Letters 92:23-31). A number of recent studies using bispecific antibody fragment constructs that simultaneously target a cancer antigen and FcαRI indicate that engagement of FcαRI can result in cell-mediated tumor cell killing (Stockmeyer et al., 2000, J. Immunol. 165: 5954-5961; Stockmeyer et al., 2001, J. Immunol. Methods 248: 103-111; Sundarapandiyan et al., 2001, J. Immunol. Methods 248: 113-123; Dechant et al., 2002, Blood 100: 4574-80). In addition, a recent study has shown that anti-FcγRI and FcαRI bispecifics in combination provide synergistic anti-tumor efficacy, indicating that simultaneously targeting gamma and alpha Fc receptors may provide a means for enhancing the anti-cancer efficacy of antibodies and Fc fusions (van Egmond et al., 2001, Cancer Research 61: 4055-4060). The structure of the extracellular domain of FcαRI has recently been solved (Ding et al., 2003, J. Biol. Chem. 278: 27966-27970), as has the receptor in complex with IgA Fc (Herr et al., 2003, Nature 423: 614-620), and the interface has been characterized with mutagenesis (Wines et al., 1999, J. Immunol., 162: 2146-2153; Wines et al., 2001, J. Immunol. 166: 1781-1789). FcαRI binds to IgA Fc at a site between the CH2 and CH3 domains, shown in FIG. 7. Notably, despite substantial structural homology between gamma and alpha Fc and between FcγRs and FcαRI, the IgA/FcαRI interaction is structurally distinct on Fc from the IgG/FcγR interaction (FIG. 2).

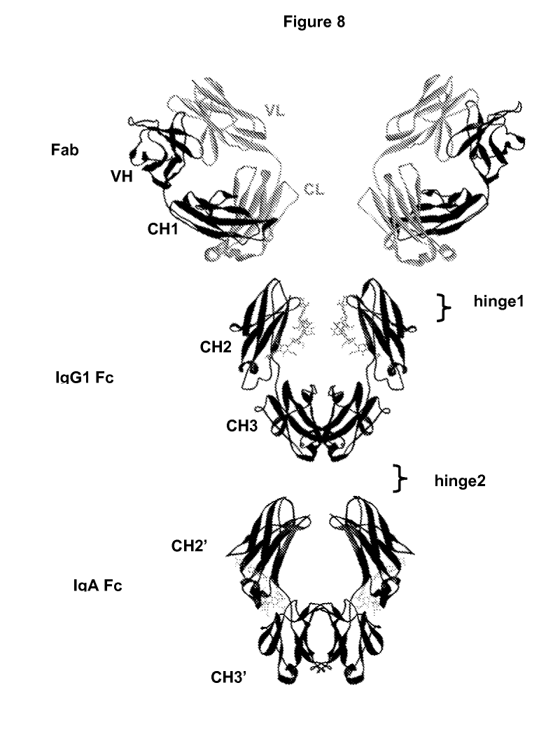

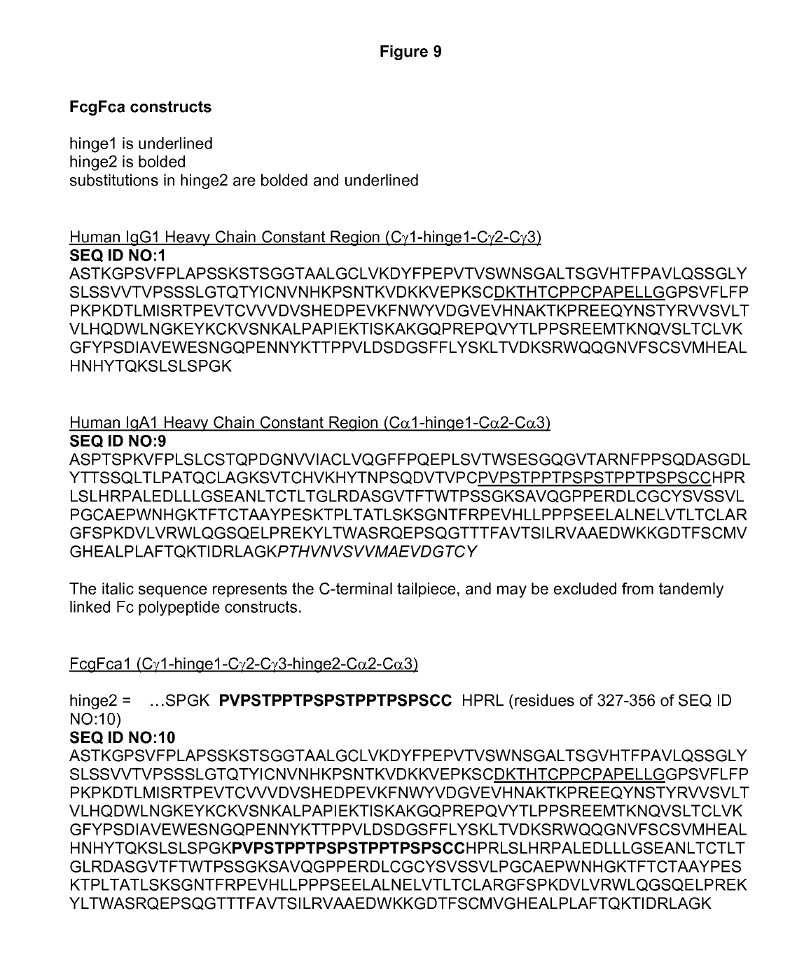

An embodiment of the present invention provides optimized Fc polypeptides wherein Fc regions from different isotypes are contiguously linked, referred to herein as hetero-contiguously linked Fc polypeptides. In a preferred embodiment, the hetero-contiguously linked Fc polypeptide of the present invention comprise Fc regions from IgG and IgA antibodies. Previous studies have been carried out, aimed towards different goals, wherein Ig domains of IgA have been engineered into IgG (Chintalacharuvu et al., 2001, Clinical Immunology 101:21-31; U.S. Pat. No. 6,284,536, Ma et al., 1995, Science 268:716-719; Ma et al., 1994, Eur J Immunol 24:131-8; U.S. Ser. No. 10/372,614). The current invention is aimed at optimizing the effector functions of antibodies and Fc fusions; due to the capacity to recruit different cell types and engage different receptors on the same cell, and because of the synergy observed using bispecifics targeting FcγR and FcαRI receptors (van Egmond et al., 2001, Cancer Research 61: 4055-4060), an antibody or Fc fusion that comprises the full Fc region of IgG and IgA, and therefore binds both FcγRs and FcαRI, may provide a significantly optimized properties. FIG. 8 illustrates a hetero-contiguously linked Fc polypeptide that comprises one Fc region from IgG Fc linked to an Fc region from IgA Fc, referred to herein as FcgFca. Here the N-terminal Fc region is that of IgG1 (i.e. CH2 and CH3 are the IgG1 CH2 and IgG1 CH3 domains respectively), and the C-terminal Fc region is that of IgA (i.e. CH2′ and CH3′ are the IgA CH2 (Cα2) and IgA CH3 (Cα3) domains respectively). Hinge1, as designated in FIG. 8, links the Fab regions of the antibody with the first Fc region, whereas hinge2 links the first and second Fc regions. Again, because the hinge regions play important structural and functional roles, it may be advantageous to explore a number of engineering constructs to obtain hetero-contiguously linked Fc polypeptides with the most favorable structural and functional properties. FIG. 9 provides a genetic construct aimed at engineering an effective FcgFca, referred to as FcgFca1. Here an IgG1 Fc region is linked at its C-terminus to an IgA1 Fc region via a hinge2 that is identical to the WT hinge of IgA1. The italic sequence in FIG. 9 (SEQ ID NOS:1, 9, 10) represents the C-terminal tail piece (tp), responsible for binding the J chain. The tail piece may or may not be excluded from contiguously linked Fc polypeptide constructs, depending on the desired goal. In alternate embodiments, this C-terminal region may be included, and may provided novel or optimal properties in the context of a hetero-contiguously linked Fc polypeptide.

FcgFca1 was constructed with the variable region of alemtuzumab, subcloned into the pcDNA3.1Zeo vector as described above, and sequenced to confirm the fidelity of the sequence. Plasmids containing heavy chain genes were co-transfected with plasmid containing light chain genes into 293T cells. Media were harvested 5 days after transfection, and the FcgFca1 protein was purified from the supernatant using protein A affinity chromatography.

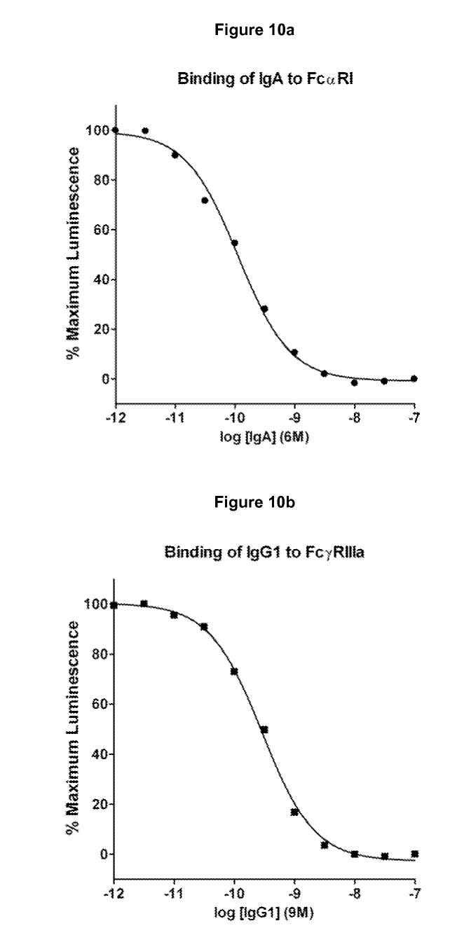

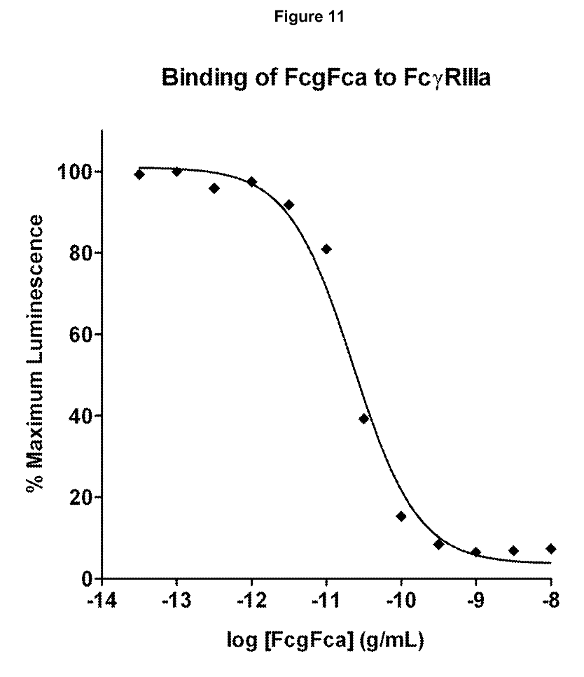

In order to screen for FcαR binding, the extracellular region of human FcαRI was fused with glutathione S-Transferase (GST). Tagged FcαRI was transfected in 293T cells, and media containing secreted FcαRI were harvested and purified. The ALPHASCREEN™ assay was used to measure binding of IgA and IgG to their respective receptors. IgA (purchased from Pierce) was biotinylated by standard methods and attached to streptavidin donor beads, and tagged FcαRI was bound to glutathione chelate acceptor beads. FIG. 10a provides dose response ALPHASCREEN™ data showing that IgA binds FcαRI. FIG. 10b shows the analogous IgG1/FcγRIIIa ALPHASCREEN™ data, obtained using biotinylated IgG (Sigma Aldrich) donor beads and GST fused FcγRIIIa acceptor beads as described above. Binding of the expressed and purified FcgFca1 polypeptide to FcγRIIIa was measured using biotinylated IgG streptavidin donor beads and GST FcγRIIIa glutathione acceptor beads as described above. FIG. 11 provides a competition assay showing binding of FcgFca1 alemtuzumab to human V158 FcγRIIIa, indicating that the FcgFca1 polypeptide maintains the binding site for FcγRIIIa. Further experimentation of these and other hetero-contiguously linked Fc polypeptides are contemplated. Preferrably, the variants are tested for binding to FcαRI. Binding studies evaluating the capacity of the Fc polypeptides to bind other Fc ligands, including but not limited to other FcαRs, complement protein C1q, FcRn, and protein A, are also contemplated. Cell-based assays may be used to evaluate the capacity of the variants to mediate effector functions. Pre-clinical and clinical experiments may ultimately be used to evaluate the potential of the variant Fc polypeptides for therapeutic use.

The contiguously linked Fc polypeptides provided in Examples 1 and 2 are not meant to constrain the present invention to these particular embodiments. In an embodiment, the present invention contemplates a variety of embodiments of the general concept of homo- and hetero-contiguously linked Fc polypeptides. Any number of Fc regions from any of the recognized immunoglobulin constant region genes, including mu (μ), delta (δ), gamma (γ), sigma (ε), and alpha (α), which encode the IgM, IgD, IgG (including IgG1, IgG2, IgG3, and IgG4), IgE, and IgA (including IgA1 and IgA2) isotypes respectively, may be linked contiguously to generate a homo- or hetero-contiguously linked Fc polypeptide. Fc regions may be linked in any order. For example, in addition to FcgFca as provided in Example 2, other embodiments of hetero-contiguously linked Fc polypeptides include FcaFcg, wherein the IgA Fc region is the first Fc region and the IgG Fc region is the second and C-terminal Fc region. Likewise, homo- and hetero-contiguously linked Fc polypeptides need not be limited to two contiguously linked Fc regions, and thus may comprise any number of Fc regions linked contiguously. IgA and IgM Fc regions may comprise their respective tail piece, and may also be bound by the J chain. Functional analogs of an Fc region, as defined above, may also find use in the present invention for generation of contiguously linked Fc polypeptides. As will be appreciated by one skilled in the art, the properties of any given contiguously linked Fc polypeptide will depend on the construct. For example, it is anticipated that because there are multiple FcRn binding sites on contiguously linked Fc polypeptides that comprise two or more IgG Fc regions (homo-contiguously linked IgG Fc polypeptides), pharmacokinetics may be enhanced. Likewise, it is anticipated that because there are multiple FcγR and C1q binding sites on contiguously linked Fc polypeptides that comprise two or more IgG Fc regions, FcγR and C1q mediated reactions such as ADCC, ADCP, and CDC may be enhanced. Likewise, it is anticipated that because there are binding sites for FcγRs and FcαRI on contiguously linked Fc polypeptides that comprise one or more IgG Fc regions and one or more IgA Fc regions, Fc receptor mediated reactions such as ADCC and ADCP may be enhanced. An array of valuable and unforeseen properties may be realized by combining Fc regions in various combinations using the concepts of engineering homo- and hetero-contiguously linked Fc polypeptides provided by the present invention.

### Example 3

### Variant Fc Polypeptides with Novel Fc Receptor Binding Determinants

In an embodiment, the present invention provides engineered Fc polypeptides with novel binding determinants, wherein one or more amino acid modifications are made in an Fc region of one antibody isotype such that it binds to an Fc receptor of a different isotype. This may be particularly applicable when the Fc binding sites for the respective Fc ligands do not significantly overlap. An example is provided whereby the structural determinants of IgA binding to FcαRI are engineered into an IgG Fc region. Notably, the IgG Fc/FcγR binding site, at the N-terminal region of CH2 and the hinge leading into it (FIG. 2), does not overlap with the structurally analogous IgA Fc/FcαRI binding site, at the interface between CH2 and CH3 (FIG. 7). Although the lack of overlap between the analogous Fc binding sites for FcγR and FcαR are not exclusive to the goal of obtaining Fc variants with novel Fc receptor binding determinants, it simplifies the engineering strategy. However, because FcαRI binds to IgA Fc at a site that is structurally analogous to the binding site on IgG Fc for FcRn (FIGS. 3 and 7) and proteins A and G, it may be more challenging to engineer IgG variants that simultaneously enable FcαRI binding but do not reduce or ablate binding to FcRn. Thus a coinciding goal may be to design Fc polypeptide variants that impart FcαRI binding into IgG1 Fc, but which do not disrupt binding to these other important Fc ligands.

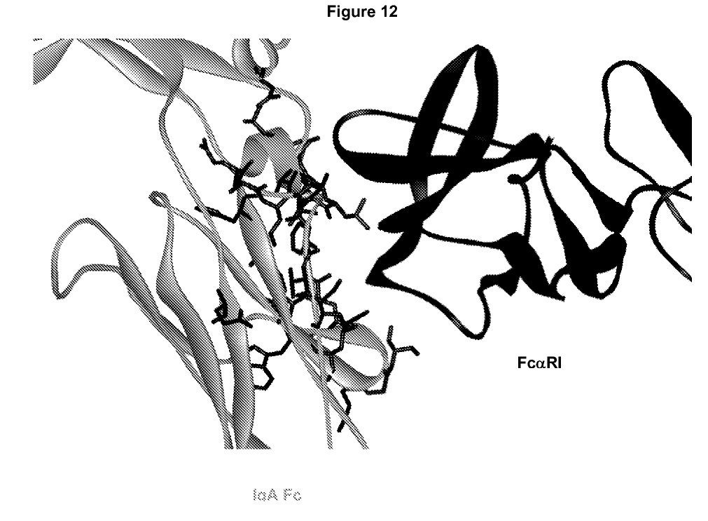

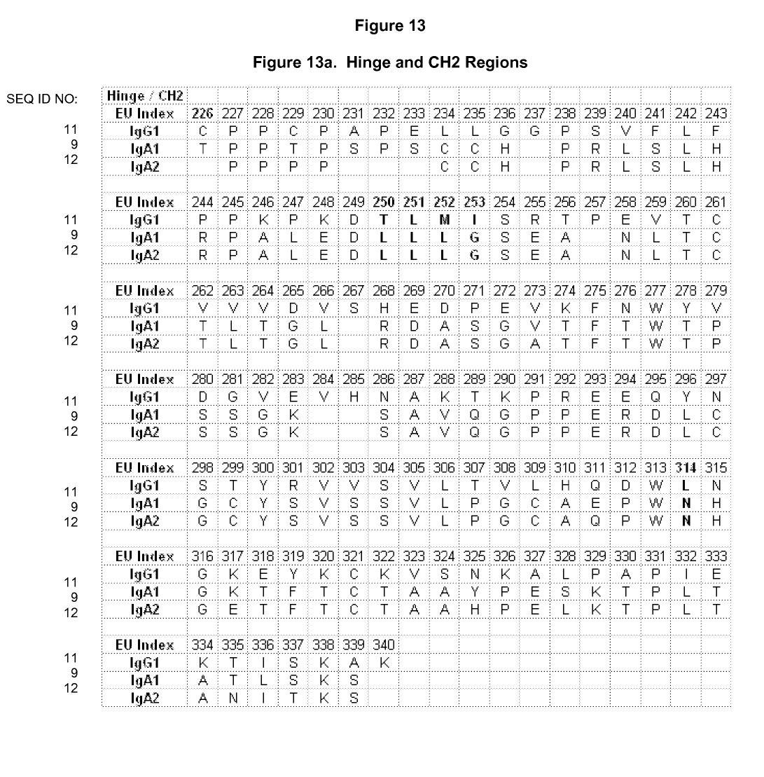

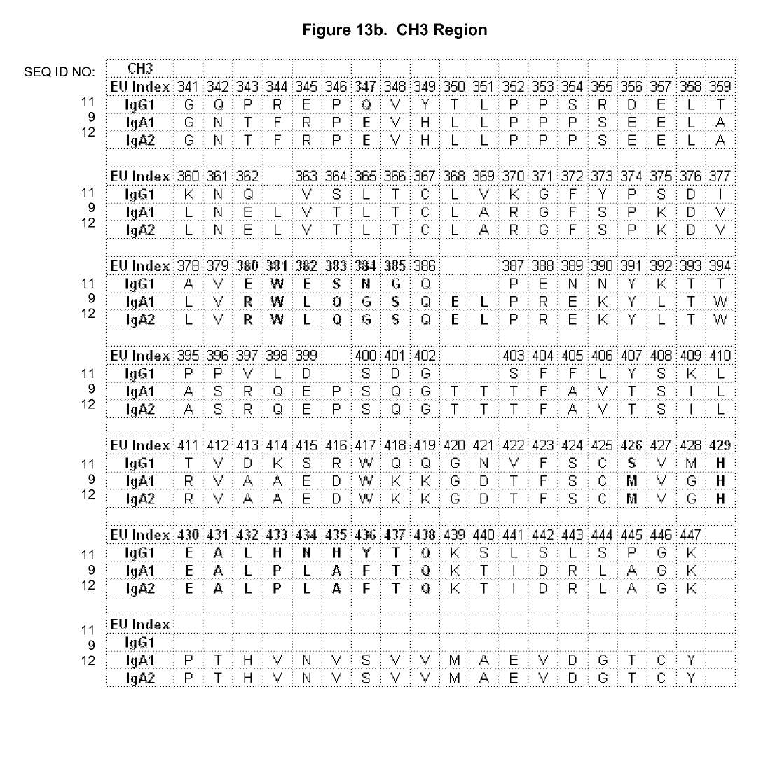

IgA residues involved in mediating FcαRI binding were identified by visual inspection of the 1OW0 structure (Herr et al., 2003, Nature 423: 614-620), and these are shown in FIG. 12. Because IgA and IgG are homologous, both structurally and by sequence, it is possible to determine the residues in IgG that are equivalent or corresponding to the FcαRI binding residues in IgA. As described above, “equivalent” or “corresponding” residues may be determined between any number of polypeptide sequences by a variety of methods known in the art. FIG. 13 provides a sequence alignment of IgA1 (SEQ ID NO:9) and IgA2 Fc (SEQ ID NO: 12) with IgG1 Fc (SEQ ID NO:11), showing IgA Fc residues that mediate binding to FcαRI and the corresponding residues in IgG1 Fc shown in bold. Here the numbering of the IgG1 sequence is according to the EU index as in Kabat. Table 1 provides the list of the IgA1 and IgA2 residues that bind FcαRI, and the corresponding residues in IgG1. In addition to the IgG1 positions (numbered according to the EU index as in Kabat), for structural reference, also provided is the IgA1 sequence numbering provided in the 1OW0 IgA1 Fc/FcαRI complex structure. EU position 386-387 indicates the presence of a deletion in the IgG sequence as compared to the IgA sequence, as shown in FIG. 13. Shaded residues in Table 1 indicate residues that are identical between IgG1 and IgA at the listed positions.

TABLE 1

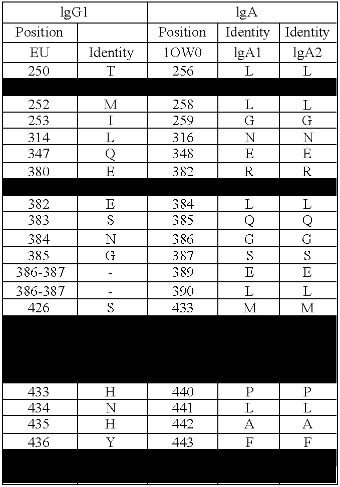

Table 1 shows that there are a significant number of identical residues between IgG and IgA at the FcαRI binding site, including L251, W381, H429, E430, A431, L432, T437, and Q438. In addition, there are a number of conserved or similar residues, including M and L at IgG1 position 252, Q and E at IgG1 position 347, and Y and F at IgG1 position 436. Thus there is already a significant degree of homology between IgG1 and IgA at the IgA residues that bind FcαRI. In an embodiment, the present invention describes IgG1 Fc polypeptides that potentially bind to FcαRI wherein one or more IgG1 residues that correspond to IgA residues that interact with FcαRI are modified to the corresponding amino acid in IgA. Thus one or more of the unshaded IgG1 residues in Table 1 may be modified to the corresponding IgA amino acid. For example, M252L, S426M, and/or N434L may be substitutions that may contribute to the engineered capacity of IgG1 Fc to bind FcαRI. Included in this set of possible amino acid modifications is the insertion of a glutamic acid, a leucine, or a glutamic acid and a leucine between EU positions 386 and 387 in IgG.

Amino acid modifications may be made individually, or may be combined in any number or manner. Thus all combinations of the substitutions listed in Table 1 are provided by the present invention. Moreover, It is not necessary to restrict substitutions in IgG1 Fc to IgA Fc amino acids. Other substitutions at any of the IgG1 residues listed in Table 1 may be explored to obtain the best variants that optimize binding to FcαRI, preferably whilst not significantly disrupting binding to other Fc ligands. In one embodiment, IgG1 variants may be engineered such that modifications are made to amino acids that are similar to the corresponding IgA amino acids listed in Table 1. For example, rather than only engineering the nonpolar to polar L314N substitution, the substitutions L314D, L314E, and/or L314Q may be explored as well. Likewise, in addition to S426M, it may be worthwhile to also explore other polar to no polar substitutions, including but not limited to S426L, S426V, S4261, S426F, S426Y, and S426W. In alternate embodiments, structure-based design or other design methods may be used to engineer substitutions in IgG1 that enable binding to FcαRI. For example, the potential for novel nonpolar FcαRI interactions at IgG position E380 suggests that in addition to E380R, it may be beneficial to explore E380L, E3801, E380V, E380F, and/or E380Y. Likewise, the potential for novel polar and charged interactions with FcαRI at IgG position G385 suggests that, in addition to G385S, it may be prudent to explore the substitutions G385D, G385E, G385N, and/or G385Q. It is not necessary to restrict substitutions in IgG1 Fc to the non-shaded positions listed in Table 1, or to the positions listed in Table 1. Residues proximal to the residues listed in Table 1, for example residues within 10 Å, preferably within 6 Å, most preferably within 4 Å, may also play a role in mediating an IgG1 Fc/FcαRI interaction, either directly or indirectly. Thus it may be worthwhile to explore modifications at residues that are distal to the FcαRI interface, including but not limited to IgG modifications to corresponding IgA Fc amino acids or to other amino acids.

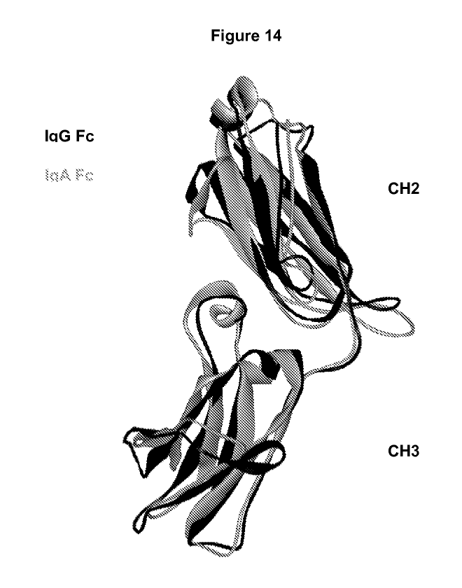

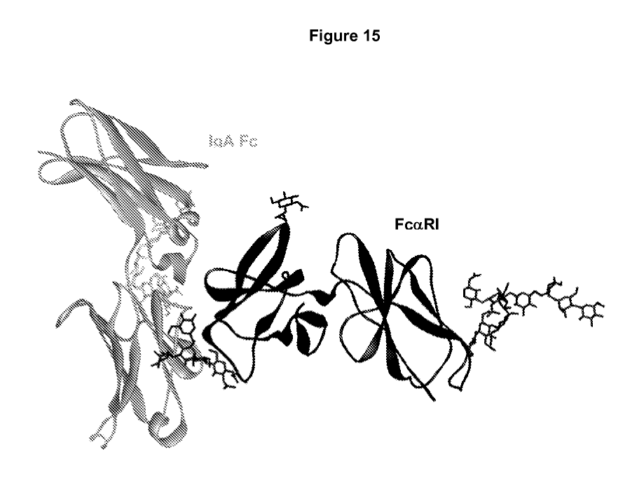

The validity of the provided engineering approach is determined not only by the sequence homology between IgG and IgA at the IgA/FcαRI interface, but also by the structural homology between the two Fc regions. FIG. 14 shows a structural superposition of the IgG1 Fc region (1DN2, DeLano et al., 2000, Science 287:1279-1283) and the IgA Fc region in its conformation as determined in the IgA/FcαRI complex structure (1OW0, Herr et al., 2003, Nature 423: 614-620). The RMSD between the backbone atoms of the superposed structures is 2.9 Angstroms, and as can be seen the overall conformations of the two Fc regions are very similar, including importantly the angle between the CH2 and CH2 Ig domains. This result suggests that replacement of IgG residues with the corresponding IgA residues is a potentially viable strategy for engineering IgG variants that bind FcαRI. An additional factor that may impact the strategy is the glycosylation of both IgA and FcαRI, shown in FIG. 15 (Herr et al., 2003, Nature 423: 614-620). There are three carbohydrates on the FcαRI receptor, one of which, attached at Asn58, plays a role in mediating binding to IgA Fc. Although IgA Fc is glycosylated at a site that is distinct from the site of IgG Fc glycosylation, the IgA Fc carbohydrate does not directly contact FcαRI. Thus the absence of an IgA Fc like glycosylation in IgG1 Fc should not preclude binding to FcαRI. Together, these results and analyses support the strategy that the FcαRI binding determinants of IgA can be engineered into IgG.

Table 2 provides a number of human IgG1 Fc variants with the potential capacity for binding FcαRI, designed based on this strategy.

TABLE 2

| Variant | Substitutions |
|---|---|
| 1 | M252L/S426M/N434L |
| 2 | M252L/S426M/N434L/E382L/Y436F |
| 3 | M252L/S426M/N434L/E382L/Y436F/H435A |
| 4 | M252L/S426M/N434L/E382L/Y436F/H435A/H433P |
| 5 | M252L/S426M/N434L/E382L/Y436F/H435A/H433P/N384G/ |
| | G385S |
| 6 | M252L/S426M/N434L/E382L/Y436F/H435A/H433P/N384G/ |
| | G385S/I253G |
| 7 | M252L/S426M/N434L/E382L/Y436F/H433P/N384G/G385S |
| 8 | M252L/I253G |
| 9 | E382L/S426M |
| 10 | N384G/G385S |
| 11 | H433P |
| 12 | H435A |
| 13 | I253G |

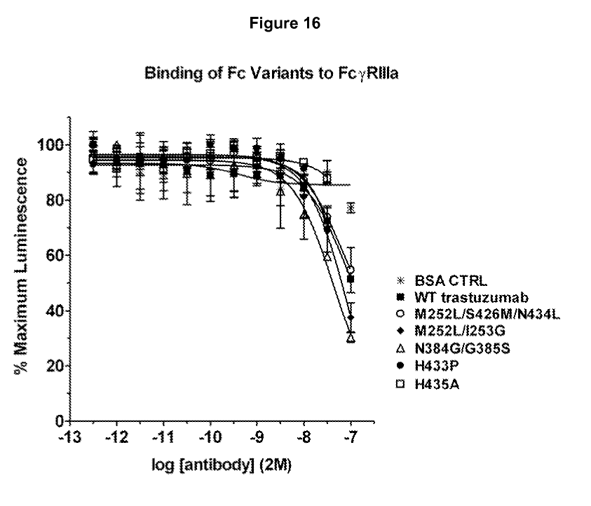

The modifications listed in Table 2 were introduced into the heavy chain sequence of the anti-Her2 antibody trastuzumab (Carter et al., 1992, Proc Natl Acad Sci USA 89:4285-4289) using quick-change mutagenesis techniques (Stratagene). Variants were sequenced to confirm the fidelity of the sequence. Plasmids containing heavy chain gene (VH-CH1-CH2-CH3) (wild-type or variants) were co-transfected with plasmid containing light chain gene (VL-CLκ) into 293T cells. Media were harvested 5 days after transfection, and antibodies were purified from the supernatant using protein A affinity chromatography. Binding affinity to human FcγRIIIa by the variant Fc polypeptides was measured using the ALPHASCREEN™ assay. The ALPHASCREEN™ assay was applied as a competition assay as described above, using biotinylated IgG donor beads and GST FcγRIIIa acceptor beads. FIG. 16 shows data for binding of select Fc variants to human V158 FcγRIIIa, indicating that FcγR binding capacity of the variants is uncompromised relative to WT trastuzumab.

A broad array of additional experiments to further test these and other variant Fc polypeptides are contemplated. Preferrably, the variants are tested for binding to FcαRI. Furthermore, as discussed, due to the significant overlap of the analogous FcαRI binding site with the IgG Fc binding site for FcRn, it will be important to determine the capacity of the variants to bind FcRn and/or protein A. Binding studies evaluating the capacity of the Fc polypeptides to bind other Fc ligands, including but not limited to other FcγRs, as well as the complement protein C1q, are also contemplated. Cell-based assays may be used to evaluate the capacity of the variants to mediate effector functions. Pre-clinical and clinical experiments may ultimately be used to evaluate the potential of the variant Fc polypeptides for therapeutic use.

Whereas particular embodiments of the invention have been described above for purposes of illustration, it will be appreciated by those skilled in the art that numerous variations of the details may be made without departing from the invention as described in the appended claims. All references are herein expressly incorporated by reference.

We claim:

1. A single polypeptide comprising two or more Fc regions linked contiguously.

2. A Fc polypeptide according to claim 1, wherein said Fc regions are of the same antibody isotype.

3. A Fc polypeptide according to claim 2, wherein said Fc regions are of an IgG isotype.

4. A Fc polypeptide according to claim 4, wherein said Fc ligand is an FcγR.

5. A Fc polypeptide according to claim 1, wherein a first Fc region is a different antibody isotype compared to a second Fc region.

6. A Fc polypeptide according to claim 5, wherein said first Fc region an IgG isotype and said second Fc region is an IgA isotype.

7. A Fc polypeptide according to claim 5, wherein said Fc polypeptide binds an FcγR.

8. A Fc polypeptide according to claim 5, wherein said Fc polypeptide binds FcαRI.

9. A Fc polypeptide according to claim 8, wherein said Fc polypeptide also binds an FcγR.

10. Isolated nucleic acids encoding a single polypeptide comprising two or more Fc regions linked contiguously.

11. A isolated nucleic acids according to claim 10, wherein said encoded Fc regions are of the IgG isotype.

12. A isolated nucleic acids according to claim 10, wherein a first encoded Fc region is a different antibody isotype compared to a second encoded Fc region.

13. A isolated nucleic acids according to claim 10, wherein said encoded Fc polypeptide binds an FcγR.

14. A isolated nucleic acids according to claim 10, wherein said encoded Fc polypeptide binds FcαRI.

15. A isolated nucleic acids according to claim 14, wherein said encoded Fc polypeptide also binds an FcγR.

16. A variant Fc polypeptide comprising one or more amino acid substitutions compared to a parent Fc polypeptide, wherein said variant Fc polypeptide substantially binds to at least one Fc ligand that is not substantially bound by the parent Fc polypeptide.

17. A variant Fc polypeptide according to claim 16, wherein said variant Fc polypeptide binds FcαRI and one or more FcγRs.

18. A variant Fc polypeptide according to claim 17, wherein said variant Fc polypeptide is an IgG Fc polypeptide that binds FcαRI.

19. A Fc polypeptide according to claim 16, wherein said variant Fc polypeptide comprises at least one amino acid modification at a position selected from the group consisting of 250, 251, 252, 253, 314, 347, 380, 381, 382, 383, 384, 385, 426, 429, 430, 431, 432, 433, 434, 435, 436, 437, and 438, wherein the numbering is according to the EU index as in Kabat.

20. An Fc polypeptide according to claim 16, wherein said variant Fc polypeptide comprises at least one amino acid modification selected from the group consisting of T250L, M252L, 1253G, L314N, Q347E, E380R, E382L, S383Q, N384G, an E insertion between positions 386 and 387, an L insertion between residues 386 and 387, G385S, S426M, H433P, N434L, H435A, and Y436F, wherein the numbering is according to the EU index as in Kabat.
